(19) United States
(12) Patent Application Publication
(10) Pub. No.: US 2024/0138745 A1
(43) Pub. Date: May 2, 2024

(54) FIELDABLE EEG SYSTEM, ARCHITECTURE, AND METHOD

(71) Applicants: David YONCE, Malvern, PA (US); Gregory KOELLER, Apple Valley, MN (US)

(72) Inventors: David YONCE, Malvern, PA (US); Gregory KOELLER, Apple Valley, MN (US)

(21) Appl. No.: 18/279,091

(22) PCT Filed: Feb. 28, 2022
(86) PCT No.: PCT/US2022/018210

§ 371 Date: Aug. 28, 2023

Related U.S. Application Data
(60) Provisional application No. 63/154,751, filed on Feb. 28, 2021.

(51) Int. Cl. A61B 5/377; A61B 5/00; A61B 5/16; A61B 5/256

(52) U.S. Cl. CPC A61B 5/377; A61B 5/0006; A61B 5/0022; A61B 5/165; A61B 5/256

(57) ABSTRACT

A fieldable EEG signal monitoring device, system and method configured for receiving and analyzing EEG signals and other user and environmental signals that is easily operate and repaired by a user and that is able to correlate received user and environmental data from one or more user to enable users or third part users to make strategic decisions about health, work, police, and military actions.

### PRIORITY

This Application is a Section 371 US National Stage Application of PCT Application No. PCT/US2022/018210, filed Feb. 28, 2022, which claims priority to and the benefit of U.S. Provisional Application No. 63/154,751, filed on Feb. 28, 2021, which is hereby incorporated herein by reference in its entirety.

### TECHNICAL FIELD

The present invention relates generally to electroencephalogram (“EEG”) systems, architectures, and methods related to measuring and monitoring subjects. More particularly, to fieldable EEG systems, architectures, and methods related to measuring and monitoring individuals such as military, police, patients, and consumer subjects outside of a typical clinical setting.

### BACKGROUND

An electroencephalograph is an electrophysiological monitoring device that is able to record electrical activity of a subject's brain. Since at least the late 1800's scientist have been recording the electrical activities of humans and animals. Electroencephalography (“EEG”) typically includes a number of electrodes that are placed on a subject, typically the head, to record voltage fluctuations/changes that occur from ionic currents within the firing neurons of a subject's brain.

It was quickly discovered that the voltage fluctuations of the brain had numerous applications. The applications included using the EEG as a diagnostic or clinical tool to diagnose conditions such as epilepsy, sleep disorders, state of consciousness, and even brain death. Even while advancements in medical technology moved forward, such as with the invention of the MRI, the EEG's ability to monitor spontaneous changes over time, cements its importance in medicine.

The electrodes of the electroencephalograph conventionally include an adhesive or paste that secures the electrode to the subject's head. Electrodes are also conventionally mounted to or coupled to a holder or substrate such as a headband or head stocking. The electrodes typically include a wire that is coupled an electroencephalograph that detects the voltage changes and prints the results or findings on a screen or piece of paper that is analyzed by a healthcare worker. Traditional electrodes also typically required the use of a gel or other medium be placed between a subject's head and the electrodes in order to improve the signal transmission.

The electroencephalograph generally consists of an electronic circuit including amplifiers and controls for processing the electrical signals received by the electrodes. The electroencephalograph also traditionally included an output device, such as an oscillograph, or more recently, a liquid crystal display, for converting the data into a readable form. All of these devices have traditionally been large, heavy, and generally required to be stationary within a room.

Various attempts have been made to provide EEG systems, architectures, and methods that can be comfortably worn by a subject. While advancements for comfortability for the subject have been made, they have failed to provide EEG systems, architectures, and methods that are needed for modern times.

What is needed and what is provided by the present invention includes having EEG systems, architectures, and methods that are easily mobile and easily used by subjects in vast or remote areas while also providing a clinical-grade signal quality having no to minimal motion artifacts. The present invention also provides EEG systems, architectures, and methods having electrodes that easily replaced or exchanged by a subject while in a remote area. Another advantage of the present invention is its ability to operate within a remote network that collects subject data in real-time. Yet another advantage of the present invention is its ability to collect individual subject data while in the subject is in the field and then can transmit, upload, or download the subject's data once the subject is in a secure area or location.

The above is not intended to limit the scope of the invention, or describe each embodiment, aspect, implementation, feature, or advantage of the invention. The detailed technology and preferred embodiments for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

What is needed is an EEG system that easily enables synchrony among a group of individuals being monitored. Typically, researchers use multiple EEG systems and manually align the data. Alternatively, traditional systems use open-source components with an IR blaster coupled with an infrared sensor to synchronize the clocks of multiple devices. The data in these situations is collected wirelessly and may also have to be manually aligned.

In the past, film and other media screenings were performed in a movie studio or screening room and viewers would record their reactions either by surveys at the end of the video/media clip or adjusting a dial indicative of the magnitude of their positive or negative feeling associate with a particular clip. Subsequent systems have used physiological sensors such as heart rate or galvanic skin response. EEG has also been shown to be useful, however, most of these systems either require a large EEG system or were subject to significant user preparation and not convenient for large prescreening studies.

Further, the 2020 COVID-19 worldwide pandemic has pushed the distribution of new video and movie content from theatres to first-time viewing over digital streaming in the people's homes. The trend towards in-home streaming of content was already growing prior to the pandemic and may reflect a permanent shift to the main method of video entertainment reception. Social distancing requirements of the pandemic have limited the ability to perform film and media screening and testing in theaters. Further, because a growing majority of video entertainment content is received via digital streaming in the home, the location of video testing and screening in a theater or other location represents a deviation in environment where the average user would view the entertainment, potentially creating a deviation in results. What is needed is an EEG system that can be used remotely to provide users relevant EEG data that can be used when marketing products or media.

### BRIEF SUMMARY OF THE INVENTION

One of the many benefits of the present invention is its ability to monitor an individual or group of individuals or subject users. One example includes the monitoring of a particular state of the groups mind or body. Various states can be monitored, including but not limited to their mental state or health, behavioral state, and health state to name of few. The monitoring of the states is important to be able to monitor how a team works together in various situations and circumstances as will now be described. The system and methods of the present invention can simultaneously monitor, collect, and synchronize EEG data and/or non-EEG from a group of individuals. One advantage of the present invention is that the system includes the ability to analyze the synchrony of a group of individuals. Synchrony is based upon the idea that group dynamics, teamwork, and human response can be better told and measured by viewing the EEG response of multiple people, users, or subjects at the same time.

The system and methods of the present invention can use the group's EEG data to determine and understand the group's response to stimuli, which could be an image such as a picture or a video; it could also be an event that the group is experiencing together, including but not limited to a concert, a movie, a gathering, or an altercation or engagement such as experienced by law enforcement or the military. The system and methods of the present invention can also use individual user data and group data to monitor a state of the group or the group dynamics, such as a degree of teamwork, group and individual fatigue, and level of group aggression to name a few.

### BRIEF DESCRIPTION OF THE DRAWINGS

In the Accompanying Drawings:

FIG. 1A is a functional diagram of the fieldable EEG system of in accordance with the embodiments of the invention.

FIG. 1B is a functional diagram of the fieldable EEG system in accordance with the embodiments of the invention.

FIG. 2A is a perspective view a sensor assembly slab of the fieldable EEG system in accordance with the embodiments of the invention.

FIG. 2B is a perspective view a sensor assembly divided into individual sensors in accordance with the embodiments of the invention.

FIG. 2C is a cross section view of an example sensor in accordance with the embodiments of the invention.

FIG. 2D is a top view of an example sensor in accordance with the embodiments of the invention.

FIG. 2E is an end view of an example sensor having peelable conductive surfaces in accordance with the embodiments of the invention.

FIG. 3 is a top view of a user's head showing example sensor locations in accordance with the embodiments of the invention.

FIG. 4 is a perspective view of a sensor coupler or housing in accordance with the embodiments of the invention.

FIG. 5 is a side view of an example sensor coupler and sensor in accordance with the embodiments of the invention.

FIG. 6A is a top view of an example sensor board in accordance with the embodiments of the invention.

FIG. 6B is a bottom view of an example sensor board in accordance with the embodiments of the invention.

FIG. 6C is an end view of an example sensor board in accordance with the embodiments of the invention.

FIG. 7A is a cross section view of an example sensor in accordance with the embodiments of the invention.

FIG. 7B is a cross section view of an example sensor held to a head accessory with a magnet in accordance with the embodiments of the invention.

FIG. 7C is a side view of an example sensor assembly in accordance with the embodiments of the invention.

FIG. 8 is a top view of an example sensor board having a plug and play feature in accordance with the embodiments of the invention.

FIGS. 9A and 9B are example head accessories in accordance with the embodiments of the present invention.

FIGS. 9C-9E are examples electrode assemblies positioned in a headband along the lines of 9A-9E and 9A-9E in accordance with the embodiments of the present invention.

FIG. 10 is a flow chart illustrating the data selection process of the fieldable EEG system of the present invention.

FIG. 11 is a front view of a head accessory on a user's head that is tethered to a smart device in accordance with the embodiments of the invention.

FIG. 12A is functional chart showing an example of media use of the system in accordance with the embodiments of the invention.

FIG. 12B is flowchart showing an example of media use of the system in accordance with the embodiments of the invention.

FIG. 12C is flowchart showing an example of product placement with the system in accordance with the embodiments of the invention.

FIG. 13A is a functional chart of operations/employee monitoring in accordance with the embodiments of the invention.

FIG. 13B is a flowchart of a fatigue score in accordance with the embodiments of the invention.

FIG. 13C is a functional chart of operator monitoring using the system and showing fatigue schematic in accordance with the embodiments of the invention.

FIG. 14 is a functional chart of a user interface monitoring using the system in accordance with the embodiments of the invention.

FIG. 15 is a flowchart of user interface monitoring in accordance with the embodiments of the invention.

FIG. 16A is a perspective view of a head accessory having status indicators in accordance with the embodiments of the invention.

FIG. 16B is a partial cross section view of the head accessory of FIG. 16A and a remote device in accordance with the embodiments of the invention.

FIG. 16C is a schematic of an electrode testing process in accordance with the embodiments of the invention.

FIG. 17A is a flowchart of electrode off detection process in accordance with the embodiments of the invention.

FIG. 17B is a flowchart of electrode validation process in accordance with the embodiments of the invention.

FIG. 18 is a perspective view of a head accessory having different sensors, a pad member, and an adjuster in accordance with the embodiments of the invention.

### DETAILED DESCRIPTION

In the following descriptions, the present invention will be explained with reference to various exemplary embodiments. Nevertheless, these embodiments are not intended to limit the present invention to any specific example, environment, application, or particular implementation described herein. Therefore, descriptions of these example embodiments are only provided for purpose of illustration rather than to limit the present invention.

Dimensions and relative proportions of components are merely example embodiments and can be varied unless specifically limited in each claim. Thus, the dimensions can be varied without departing from the scope of the invention.

The present invention illustrates devices, systems, and methods for monitoring, analyzing, and reporting neural activity by detecting, collecting, and analyzing electroencephalogram (“EEG”) readings from individuals or groups of individuals for a number of purposes that are examined herein. The present invention can use monitored EEG readings alone or in combination with non-EEG data from other sources, including but not limited to, user anatomical data such as vital signs (e.g., blood pressure, body temperature, pulse rate, respiration rate, heart rhythm), and anatomical changes, (e.g., eye movement, muscle twitches, facial movement, perspiration). Other non-EEG data that can be used includes environmental stimuli (e.g., photos, movies, commercials or ads, concerts, large gatherings, or police and military encounters). The present invention can collect any observable stimuli, combine it with collected EEG data, analyze it, and provide an output that can be used by users, clinicians, marketing companies, companies with employees, and the military and police.

### System Architecture

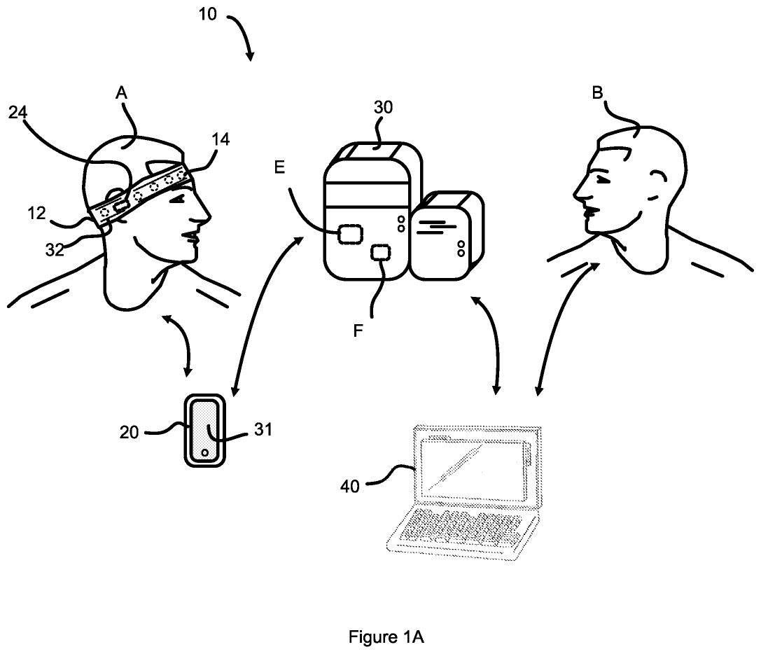

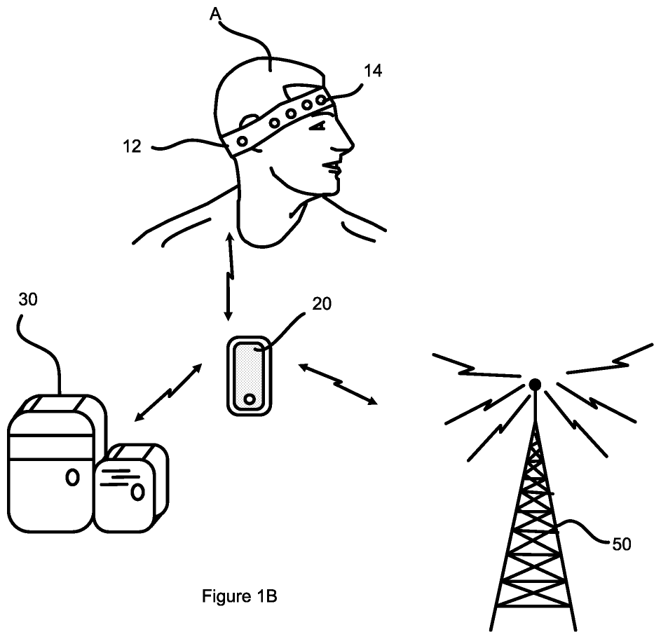

In its simplest form, the system 10 of the present invention comprises different components or parts. As illustrated in FIGS. 1A and 1B, the system 10 comprises at least one EEG electrode support or applicator 12, such as a headband head accessory that is securable to or about a user's head A. The EEG electrode support 12 can take any form, including but not limited to a soft headband, a helmet, or a clip. The EEG electrode support 12 may comprise any material or combination of materials. For instance, a foam or rubber material may be used alone or in combination with a generally more rigid shell.

The EEG electrode support, applicator, or assembly 12 comprises one or more sensors or sensor assemblies 14 that are capable of reading at least EEG signals. The sensors 14 are spaced apart along an inner surface 16 of the EEG electrode support or applicator 12. One of the sensors 14 is positionable proximate a user's mastoid bones and the other sensors are positioned against a user's forehead. Placement location of the electrodes 14 is only limited by the needs of the part of the user's brain needing to be monitored.

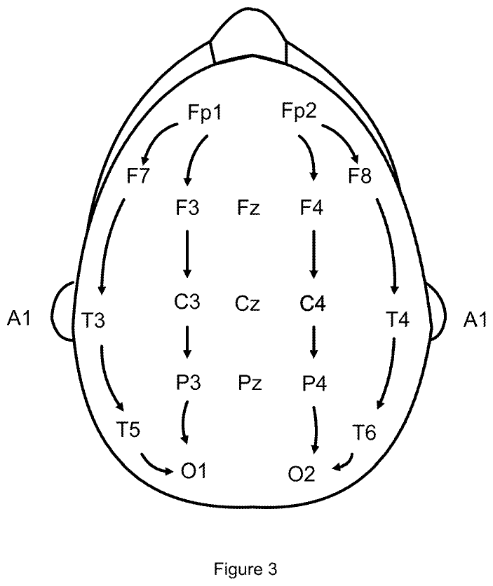

FIG. 3 illustrates example sensor 14 locations on a user's head. One or more center electrodes can be placed on the subject or user's forehead. The center electrode(s), typically designated Fpz, connects to and is in operative communication with an active bias of a bio-signal amplifier to provide a balanced noise rejection one either side of the subject or user's skull. The mastoid electrodes, identified generally by A1 or A2, provide a far field reference for the center forehead electrode(s) and also allow for monitoring of EEG signals across the two sides of the subject or user's head. This configuration allows for high ease-of-use, high signal integrity, and enables many EEG applications. Other locations include temporal (designated generally by T #), occipital (designated generally by O #), parietal (designated generally by P #)

The system 10 of the present invention also includes a remote (e.g., mobile) application device 20 that provides an alternative, yet important, function to aid in collecting EEG data and/or non-EEG data. The remote application device 20 comprises any type of smart device (e.g., smart phone, smart watches, tablets, and the like). The remote application device 20 includes wireless and wired communication assemblies (e.g., Bluetooth and WIFI) that can communicate with at least the EEG assembly 12. The remote application device 20 also includes storage capable of storing EEG data and non-EEG data. In one example embodiment of the present invention, the remote application device 20 includes a program or application that is able to analyze the EEG data and non-EEG data to determine psychologic and/or medical state of the user or users being monitored. The remote application device 20 may also be able to provide periodic trending over time.

Continuing with FIG. 1A, in an example embodiment of the system 10, a data-platform (e.g., sever or servers (fixed or cloud-based)) is included that is able to communicate wirelessly or by a physical connection with the remote application device 20. The data-platform 30 comprises at least one storage medium and random-access memory (“RAM”) that is capable of receiving, storing and analyzing the EEG data and non-EEG data. The data-platform 30 also comprises a processor that is able to perform the analysis of the EEG data and/or non-EEG data and to prepare an output or read-out of datapoints that can be correlated or that correspond with a user's state or condition. The data-platform 30 includes one or more algorithms that may be HIPPA-compliant and future predictive processing.

The system 10 is able to detect various user states or conditions, including but not limited to a medical condition, a mental state (e.g., anxiety or depression), a physical state (e.g., alertness, exhaustion, or illness), or an emotional state (e.g., happy, sad, enjoyment, like, or dislike). While examples of user states are described herein, the present invention applies to any user state and therefore the described user states should not be considered limiting.

The data-points or read-out can be sent to a clinician portal 40, such as a hospital workstation, tablet, or smart device. A clinician B is able to review the data-points or read-out and determine a course of action or treatment. The clinician portal 40 is capable of storing long-term data results to allow the clinician B to identify trends, triage based upon received data-points/trends, tailor a treatment plan based upon the long-term data and the predictive trend.

In other example embodiments of the invention, as will be discussed in more detail below, a third-party such as a logistics professional, coaches, sergeants or generals, police captains, the like may be able to use the data-points or read-outs in order to determine action of one or more user's being monitored. For example, the third-party would be able to use the data-points or read-outs to strategize movement of one or more of the monitored users A, pull one or more of the monitored users A out of the field, and the like.

The EEG head accessory 12 can take any number of configurations. In one example embodiment of the present invention, EEG head accessory 12 comprises a flexible substrate such as a headband or stocking that is able to support polymer electrodes. The EEG head accessory 12 can be manufactured from a material that mimics the mechanical properties of human skin. The EEG head accessory 12 can also be operatively coupled to or integrated into another device such as a helmet. The electrodes 14 of the present invention can also be used separately from the EEG head accessory 12 and either placed on a subject or incorporated into another garment or appliance worn by the subject or user A. For example, a subject's or user's A hat, stocking cap, helmet, headphones, glasses, headscarf, and the like.

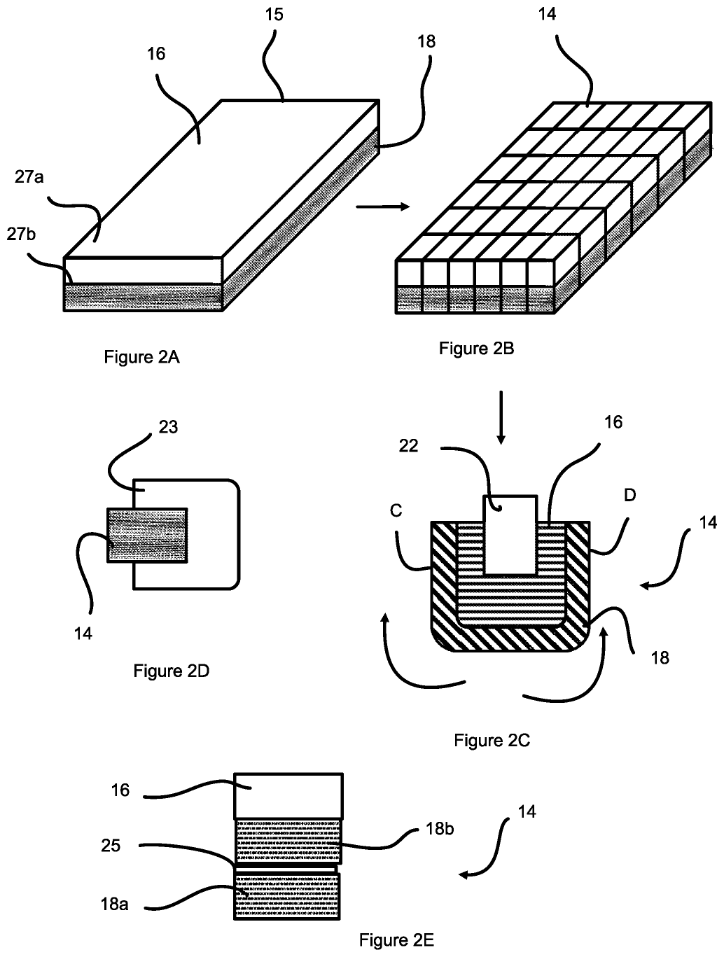

An important aspect of the present invention are the novel sensor or sensor assemblies 14. FIGS. 2A-2C illustrate example methods or process of making/manufacturing the sensors 14. As illustrated in FIG. 2A, one or more sensors 14 can be manufactured by creating a sensor bed 15 comprising a support layer 16 and a conductive layer 18. The conductive layer 18, comprises an EEG conductive material such as a composition of silver nanowires embedded in a Polydimethylsiloxane (“PDMS”), is deposited or formed. Next, the support layer 16 of PDMS is deposited on the conductive layer 18 to create a large single novel sensor electrode bed 15. This unique composition and process is able to be quickly set-up, have a high signal quality, and include a high rejection of motion artifacts for usage in nearly any environment (e.g., sitting, walking, running, and altercations or combat). The support layer comprises a first surface 27a and a second surface 27b.

As illustrated in FIG. 2B, the large sensor or bed 15 is able to be divided or separated into individual sensors 14 by using any cutting or dividing method known to one skilled in the art. The sensors 14 can be divided into any size, shape or configuration and is only dependent on the needs of the clinician, third-party, and/or the user being monitored.

Turning to FIG. 2C, a sensor 14 can be folded over another material, such as a compressive member 22 (e.g., foam, rubber, and the like). In this embodiment, the PDMS layer 16 is positioned against the compressive member 22 such that the conductive layer 18 is outwardly facing on at least two sides. One of the sides, identified as C, is positionable against a user's head, while the side, labeled D, is positionable against the head accessory 12, including any circuitry.

In another example embodiment, as illustrated in the top view of FIG. 2D, an electrode 14 is fixed or removably coupled to a compressive member 22 with the electrode 14 extending beyond the compressive member 22 so as to be able to contact a user's head. The compressive member 22 can be coupled (fixed or removable) to the head accessory 12.

An advantage of these sensors 14 and their method of manufacturing is that there is generally no wire or connection coupled to the opposed conductive layers 18 and extending through the PDMs layer 16. This greatly increases the efficiency of manufacturing while reducing areas of possible defects.

In another example embodiment of the present invention, in addition to or in lieu of, the support layer 16, a circuit substrate 17 can be adhered to or coupled to the head accessory 12 to support the various electrode 14 configurations of the invention. The circuit substrate 17 can be generally planar and be generally flexible to allow contouring with the head accessory 12. While being described as being generally flexible, the circuit substrate can also be generally rigid or a combination thereof.

Referring back to FIG. 1A, in an example embodiment, the head accessory or assembly 12 includes storage 24 that is able to store local user EEG and non-EEG data. The storage 24 can comprise any known storage device such as a hard drive, flash drive, RAM, and the like. The present invention also comprises EEG head accessory or assembly 12 that can provide real-time streaming of EEG and/or Non-EEG data over Wi-Fi, Bluetooth, or cellular towers 50 (see FIG. 1B). The communication can be either directly to the internet or to the remote application device 20, such as a user's mobile phone.

In another example embodiment, the support layer 16 can include or comprise electronic circuitry to store the EEG data or non-EEG data generated by the electrodes 14 or other sensors (e.g., cameras, thermometers, pulse rate or heart rate sensors) built either into the support layer 16 or into a shell or covering, such as a helmet that is able to communicate with the EEG head accessory or assembly 12.

To send the EEG data and/or non-EEG data, the support layer 16 can comprise, incorporate, hold, or store, a wireless transmitter E that is able to transmit the data wirelessly to the remote application device 20, which is then able to transmit the data or results to the data-platform 30, or to any other data storage device, generally identified in the drawings as letter F. The data platform 30 can comprise a hard drive disk, stick, module, or chip that is operatively coupled to or in operative communication with the support layer 16 and/or remote application device 20. The data store device E can comprise any electronic circuitry capable of storing data. In this embodiment, the storage device F may be required to either be directly connected to the support layer 16 or to be within a very short distance to the support layer 16 or assembly 12. A distance requirement or limitation can be implemented into the system to aid in reducing an ability of the EEG data or non-EEG data being nefariously captured.

The system of the present invention can also include an interactive module or application 31, such as a mobile phone software application, to facilitate interaction between a user A and the EEG head accessory 12. Through the interactive module or application 31, a user A can power on/off the device 20, monitor a control state of each component (e.g., an impedance and/or connection of each sensor electrode (leads-off sensing)(discussed below), and also stream or control the dissemination of the EEG data from the EEG head accessory 12 (e.g., from the substrate layer 16 of the headband to a mobile device 20) and/or the non-EEG data.

A monitor user B can be anyone, including but not limited, a doctor, a strategy analyst, psychiatrist, a marketing analyst, clinician, third-party, and the like. There can also be multiple monitor users B, with each monitor user B being able to control one or more features of the system 10. For example, the subject user A wearing the EEG head accessory, appliance, or assembly 12 may be able to turn on or repair one or more components of the system 10. A monitor user B can also comprise a clinician that is able to receive either raw EEG data and/or non-EEG data, or outputted datapoints for the purpose of treating the subject user A wearing the EEG head accessory or appliance 12. Other uses and users shall be described in more detail below.

As will become apparent, the present invention is able to conduct data processing of EEG data and/or non-EEG data in real-time on the interactive module or application 31, or on any device that is capable of receiving and allowing data analysis and/or control or manipulation. The interactive module or application 31 can be static or dynamic. For instance, the interactive module 31 can comprise an application on a smart phone (such as the remote application device 20) when low latency is required. The smart phone or remote application device 20 can then serve as a gateway to send EEG data or non-EEG data to the data-platform 30. The remote application device or smart phone 20 can also provide feedback or a notice to the subject user A or monitor user B on EEG trending or results calculated on the remote application device/smart phone 20, or on the data-platform 30. The interactive module or application 31 can alert a subject user A or monitor user B of any EEG monitored parameters or output datapoints, including but not limited to, a sleep/awake state, a fatigue state, a state of awareness, an anxiety level, a brain injury, a state of equilibrium, or any brain impairment or abnormality.

As mentioned above, the data-platform 30 can be a server-based, secure system for managing the collected EEG data or non-EEG data, and any determined or formulated datapoints (collectively “sensitive data”) from one or more subject users A. The sensitive data can be separated into one or more accounts for each subject user A. This improves security of the sensitive data while also ensuring compliance with medical requirements such as HIPAA.

EEG data can be collected as raw EEG data that is stored on the data-platform 30. The data-platform 30 comprises one or more algorithms that enable a number of analyses or processes, including time-frequency spectrum analyses to provide analysis back to the subject user A or caregiver. A subject user A can access the EEG data, non-EEG data, or outputs through the remote application device 20. Alternatively, or in addition to, the EEG data, non-EEG data, or outputs can be accessed by a caregiver, researcher, analyst, or other third-party monitor user B through a third-party or provider portal 40. The third-party user can obtain and examine multiple users' EEG data, non-EEG data, and/or outputs through the provider portal 40, which may comprise a laptop computer, desktop computer, tablet, smart phone, smart watch, and the like. Additionally, the EEG data, non-EEG data, and/or outputs can be deidentified and processed together with larger sets of data, machine learning, or AI algorithms to find alternative patterns or relationships. EEG data, non-EEG data, and/or outputs can be synchronized later or in real-time using the methods or steps provided below.

Polymer Electrode with Shield and Clamshell Connector

The present invention also has the novel advantage of permitting a subject user A to easily and quickly replace any electrode 14 of the EEG head accessory 12. The electrodes 14 of the present invention can be quickly replaced while in the field allowing monitor users B to continue to receive real-time EEG data and non-EEG data from all subject users A.

In one example embodiment, multiple alternating layers of PDMS 16+Silver Nano Wire 18 are employed and removable from each other to allow a user in the field to remove an outer layer that may become damaged or dirty. The different layers can be connected, coupled or adhered together by any fastening means or mechanisms. For example, an adhesive can be placed between the alternating electrode layers.

As illustrated in FIG. 2C, when an electrode 14 wrapped around the foam or compressive member 22 of the headband 12, the Silver Nano Wire or conductive layer 18 of the electrodes 14 comprises at least two surfaces: a subject user A contacting surface C and a headgear or headband 12 contacting surface D. The subject user A contacting surface C contacts the user's A skin to receive EEG signals. The headgear 12 contacting surface D can contact a conductive member or strip 32 such as a piece of metal connected to or incorporated into a portion of the headgear or head band 12. The conductive member or strip 32 can extend about an inner surface of a helmet or other headgear 12. In this way, the conductive member or strip 32 interconnects at least each of the electrodes 14 of the system 10 that are each wrapped around the foam or compressive member 22. This eliminates a need for interconnecting wires between the electrodes 14.

As illustrated in FIG. 2D, in another example embodiment of the invention, each electrode 14 has its own backing or other supportive member 23 that provide some structure to the electrodes 14. The backing or supportive member 23 can comprise any material such as a compressive material 22 like a foam or rubber material of the headband 12, the supportive layer 16, or the circuit substrate 17. In some embodiments, the electrodes 14 are able to fold about the backing or support member 23 in a similar fashion as described above. In this way, the backing or supportive member 23 can be connected to the headband or head gear 12. Any type of connecting mechanism or device can be used. For example, an adhesive, snaps, hook and loop fasteners, magnets, and the like.

### Electrode Coupler

In another example embodiment of the present invention, as described above and as illustrated in FIG. 2E, the electrodes 14 have one or more conductive layers 18a that can be separated by a cover or backer 25 to expose new electrode conductive layer 18b, the ability to replace or repair the system in the field is one important aspect of the present invention. Electrodes have a finite lifetime and will need to be replaced by novice users in the field. As such, the present invention enables novice users to replace a defective or dirty electrode 14.

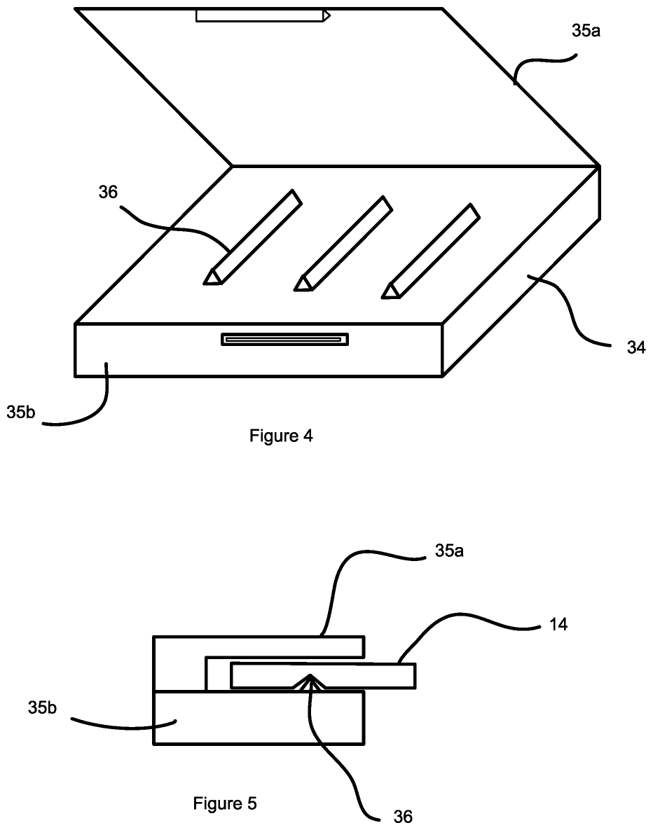

In one example embodiment, as illustrated in FIGS. 4 and 5, the system 10 comprises one or more electrode couplers or housings 34 coupled or attached to the headband member 12. The electrode couplers 34 can comprise a clamshell configuration having a lid portion 35a hinged to a base portion 35b. The lid portion 35a is configured to close onto an electrode 14 positioned between the lid portion 35a and the base portion 35b. The lid portion 35a can be configured to compresses the electrode 14 into electrical connectors or electrode sensor contacts 36 positioned in the base portion 35b of the electrode coupler 34, thereby maintaining high electrical conductivity to the circuit and also providing a secure mechanical fit. FIG. 4 shows a diagram of such an electrode coupler or connector 34. As can be seen, this example embodiment comprises multiple sensor contacts 36 within one connector 34. The sensor contacts 36 in the base portion 35b of the electrode coupler 34 can be in operative communication with wires or other conductive material coupled to or positioned in a portion of the headband member or headgear 12. As particularly illustrated in FIG. 5, a support layer 16 or compressive material 22 is not required but can be included to provide support and comfort.

As illustrated in FIG. 5, the sensor contacts 36 of the electrode coupler 34 can sit slightly above a surface of the base portion 35b of the electrode coupler 34. The electrode coupler 34 and sensor contacts 36 can be manufactured to have a biasing or a spring action whereby the sensor contacts 36 are able to move at least partially in and out of the base portion 35b of the electrode coupler 34. In one embodiment, at least a portion of the polymer electrode 14 would be fed into or placed between the lid portion 35a and base portion 35b of the electrode coupler 34. The lid portion or cover 35a would close, and alternatively latch, over at least a portion of the electrode 14. As particularly illustrated in FIG. 5, applying pressure to the lid or cover 35a causes the lid or cover 35a to compress the polymer soft electrode 14 and maintain electrical connection to the sensor contacts 36.

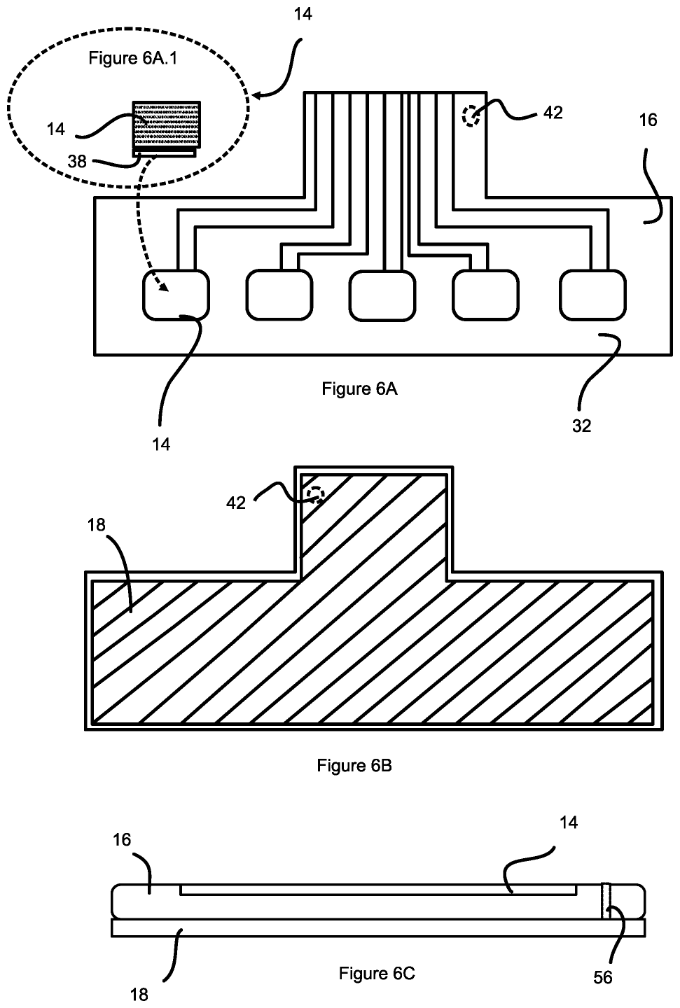

In another example embodiment of the present invention, as illustrated in FIGS. 6A-6C, a unitary or generally unitary electrode coupler 32 is provided that permits easy replacement of all electrodes 14 at the same time. The unitary electrode coupler 32 comprise a generally planar supportive member that may comprise the supporting layer 16, a connecting member 23, or the like. The electrodes 14 are coupled to, embedded on or within the coupler 32 whereby all of the electrodes 14 can be easily replaced by replacement of the coupler 32.

In another example embodiment the electrodes 14 can be coupled to rather than fixed to the coupler 34. As illustrated in FIG. 6A.1, the electrodes 14 include a fastener 38 such as a conductive adhesive layer (with or without a backer or cover 25) on at least one of the electrode 14 surfaces to allow it to be removably adhered to a contact sensor 36 of the coupler 32 and the coupler 32 can be coupled to a portion of a headband member or headgear 12.

In another example embodiment, the coupler 32 is able to make contact with a contact sensor 36 or other type of conductive member of the headband 12. When the coupler 32 is coupled to the headband member or headgear 12, conductive components on the electrode 14 and the sensor contacts 36 are able to transmit signals through headgear 12, whereby EEG signals are able to be transmitted from the electrodes 14 to the electrode coupler 32. The electrode coupler 32 can comprise other electronic circuitry to enable the function of the headband or head accessory 12. The electrode coupler 32 can be manufactured by lithography or similar manufacturing process.

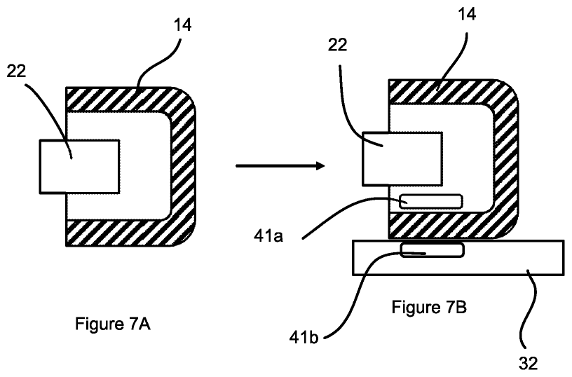

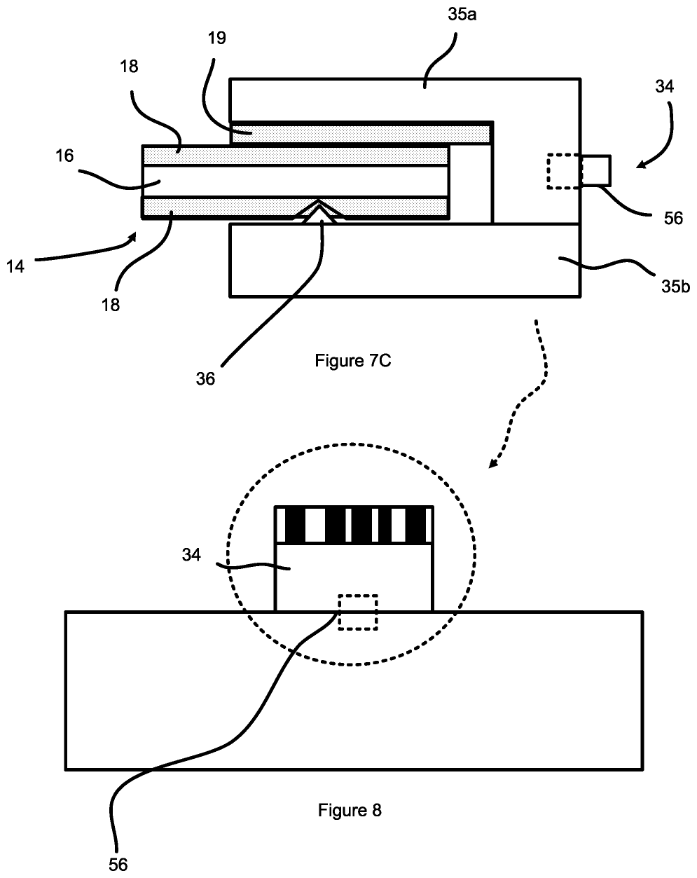

In another embodiment, as illustrated in FIGS. 7A and 7B, either one or both of the electrode 14 and headband member or headgear 12 have a magnet member 41a and 41b that enables magnet coupling to the headband member or the headgear 12. Similar to other embodiments, when the electrode 14 is placed or magnetically coupled to the headband member or headgear 12 conductive material on or in the electrode 14 are coupled to or mate with sensor contacts 36 in the headband member or headgear 12 or another portion thereof. The magnet member 41a or 41b may be encased or manufactured to include conductive material such that they act as a conductor of the EEG data and/or non-EEG data. Inside the folded electrode 14 can also be a magnet member 41a or 41b that can be conductive or non-conductive.

### Shielded Polymer Electrode

The system 10 of the present invention also comprises a fully shielded electrode 14 for use in some situations. The shielded electrode 14, as illustrated in FIG. 7C, comprises a secondary ground layer or member 42 on the back side surface of the electrode 14. The secondary ground layer or member 42 is configured to shield along at least a portion of, or an entire external or outer surface of, the electrode 14, shielding all electrodes 14 and traces from external radio frequency (“RF”) energy. When connected to a ground or an actively driven bias, this provide a biosensing system (e.g., EEG, ECG) with the highest possible level of shielding and more than is available in with commercially available electrodes today. In one example embodiment, a PDMs or other supportive layer 16 may be placed between the electrode's 14 conductive layer 18 and the ground layer 42.

FIGS. 6B and 6C show a diagram of a multi-channel electrode 14 of the system 10 that comprises a multi-layered electrode including a shield layer or member that may comprise conductive layer 18 on its back side surface. As discussed above another shield layer or member 18 can be connected or coupled to a top side surface of a layer of nano wires or conductive layer 18 with a ground connection or via 42 connecting the two conductive layers 18. This configuration provided a fully shielded electrode 14. Alternatively, if an electrode coupler 32 is utilized, it is possible to have the top of the electrode coupler 32 be conductive and connect to the ground layer or member, negating the need for a coupler 32 or to simplify the manufacturing process. Alternatively, the ground layer or member may be covered with PDMS to insulate that ground layer or member electrically from the surroundings. The grounding layer may comprise a layer of the conductive layer 18. Other materials may also be used.

As illustrated in FIG. 7C, another shielded electrode 14 of the present invention is depicted. This shielded electrode 14 does not include a coupler 32 or the ground connection 42. Instead, a novel electrode coupler 34 comprising a conductive layer 19 on either the lid or cover portion 35a or the base portion 35b, makes contact to the shield layer or member 18. This configuration minimizes and/or simplifies the electrode 14 manufacturing process while providing high electrical conductivity and mechanical robustness within the electrode coupler or connector 34. Alternatively, an electrical sensor contacts 36 or series of electrical sensor contacts 36 can be employed on the lid or cover portion 35a of the coupler or connector 34. The electrical contacts can run in any direction and can have any configuration.

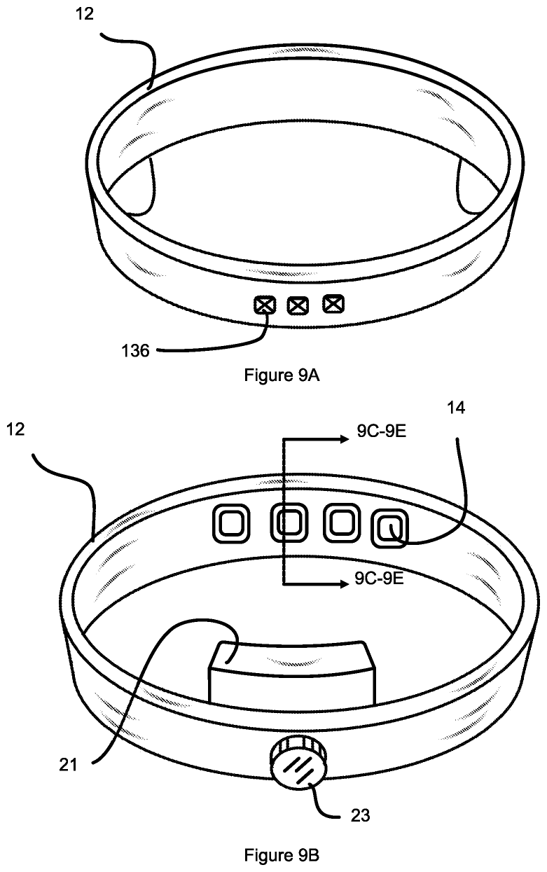

As illustrated in FIG. 8, the system 10 of the present invention also includes removable or replaceable plug-in electrodes 14. The body of the plug-in electrode 14 can comprise any of the above configurations, including having an electrode coupler housing 34 that is generally planar, or clam shaped. The electrode coupler housing 34 is configured to have a connector 56 that may comprise either a male plug or a female socket. The headband member or headgear accessory 12 can include a corresponding male plug or female socket that is configured to mate with the male or female portion of the electrode coupler housing 34. The electrode coupler housing 34 can be either sealed or it can be selectively opened and closed. This configuration of an electrode 14 allows for a more rigid and more traditional connector form-factor, for example similar to an Apple lightening connector or a micro-USB connector, to be integrated into the electrode 14 and system 10. This configuration allows a user A to repair a defective or worn electrode 14 by simply unplugging the bad electrode 14 and plugging in a new electrode 14. In this manner, the permanent termination would be integrated into the electrode 14 and the complete assembly disposed of when time to replace the electrode 14 in the headband or headgear 12.

It should be noted that the electrode coupler 34 can be configured in a variety of ways, including a flexible tail from the electrode(s) 14 that would allow it to fold over the backside to minimize the electrode 14 height. Other additional features such as the shielded and 3D electrode 14 sensor contacts 36 could be included as well in this configuration.

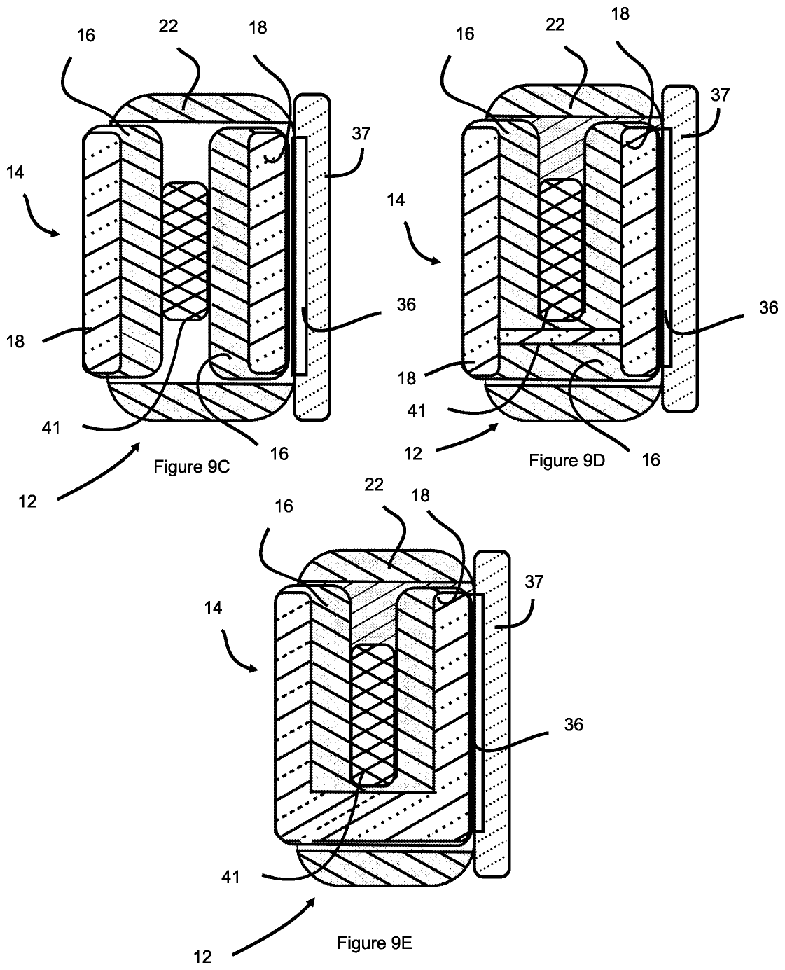

As illustrated in FIGS. 9A and 9B, the head accessory 12 comprises a generally soft yet protectable headband. As particularly illustrated in FIG. 9B, the headband 12 can include a pad member 21 positioned on the back inner surface of the headband 12. The pad member 21 is positionable on a rear portion of a user's A head. An adjuster 23 is operatively coupled to the pad member 21 and extending through the headband 12 to allow movement of the pad member 21 with respect to the headband 12. In this manner, the user A is able to adjust the headband 12 to fit their head.

Referring to FIGS. 9C to 9E, example electrode 14 embodiments are illustrated. Each of the Figures are cross sections along the lines of FIG. 9B but showing different electrode configurations. FIG. 9C illustrates two separate conductive layers 18 with PDMs layers 16 (which can be optional) held together by a magnet member 41, all of which are positioned in an aperture in the compressive material 22 of the headband 12. The magnet member 41 keeps the electrode 14 in the aperture and against the sensor contact 36 of the connector member 37.

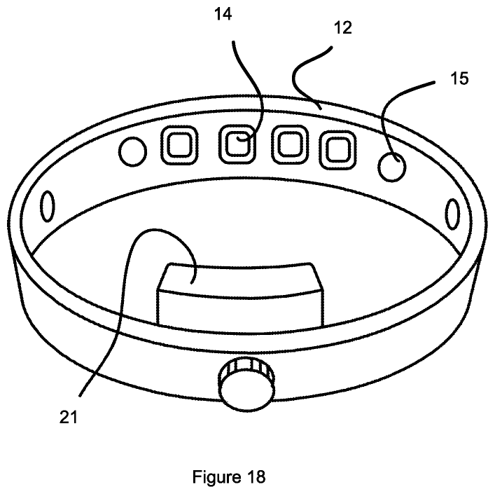

FIG. 9D illustrates a similar electrode 14-headband 12 configuration as FIG. 9C but includes a conductive bridge 33 that is coupled to each of the conductive layers 18 and extends through the PDMs layer 16. The purpose of the conductive bridge 33 is to enable the conductive layer 18 against a user's head to transmit EEG signals to the inner conductive layer 18 proximate the sensor contact 36. FIG. 9E, is similar to the folded electrode 14 configuration discussed above. FIG. 18 illustrate additional non-EEG sensors 15 incorporated into the headband 12.

### EXAMPLE APPLICATIONS

### Health Monitoring in an Individual

The system and method 10 of the present invention can be used in monitoring the health of an individual or group (as discussed later). Any aspect of heath can be monitored, including but not limited to mental health, behavioral health, and physical health. For instance, in the monitoring and/or treatment of patient-users or subject users A. In particular, it can be used to identify precision biomarkers grounded in neural circuit computations that can measure and trend a mental health condition, such as anxiety or depression, at an individual level or at a group level. The system and methods 10 provide mental health clinicians B and patient-users with EEG data, non-EEG data, or output datapoints that can be quantified and trended over time to show treatment progression or regression similar to how temperature is utilized as a monitoring measure for a flu patient or how blood pressure is used as a monitoring biomarker for a heart failure patient.

In one example embodiment of the present invention, mental health conditions such as anxiety and depression can be measured and trended utilizing a gamified algorithm or application 31 and concomitant EEG measurements. The gamified algorithm 31 entails a simple decision-making game, application, or process, to detect, measure, and determine the neural correlates of anxiety, depression, or any mental health conditions wanting to be measured or monitored.

The system and method 10 of the present invention can incorporate the gamified algorithm or decision-making game 31 into the remote application device 20 or any device having a viewable monitor or screen, such as a computer with a monitor, a television, a smart phone, and the like. The gamified algorithm or decision-making game software 31 can take a number of forms, including having an avatar that is controlled by the patient-user A. The patient-user A controls the avatar to forage for food, for example, berries in a berry patch. In order to maximize an overall reward in the form of total collected berries, the patient-user A must make “patch-leave” decisions of staying to collect the few remaining berries in the current patch as supply decreases versus the cost and “potential benefit” of moving to another berry patch. While individuals were near optimal in their foraging strategy, as prescribed by the normative model like marginal value theorem, individual differences in patch-leave decisions have been found to be indicative of an underlying brain state. What has been found is that individuals with momentarily higher levels of anxiety due to a stressor spent less than the prescribed time in a berry patch. However, those with chronically high levels of anxiety—operationalized as trait anxiety which is a precursor to clinical anxiety and depression—spent more than the prescribed time. Thus, game behavior and the gamified algorithm or decision-making game 31 can distinguish momentary anxiety (which is generally short in duration and generally caused by an adverse event or situation, such as PTSD) from trait anxiety (which is generally considered to be a trait of a person's personality).

In a conventional gamified algorithm session, without the system and methods 10 of the present invention, individuals with trait anxiety can be identified with approximately 60% accuracy. However, when gamified algorithm or decision-making game 31 is combined with the system and methods 10 of the present invention, which provides high-quality source localized EEG signals, the accuracy increases to approximately 85%. The system and methods 10 of the present invention can use the EEG frequency content and signal power to measure a patient-user A during the gamified algorithm or decision-making game software 31 and compare the readings with known templates from persons with sub-clinical and clinical levels of anxiety or depression. While anxiety and depression have been discussed, it should be appreciated that any mental health condition is within the spirit and scope of the present invention.

Another novel approach is to use the system and methods 10 of the present invention to monitor subject users A over a period of time to detect neural changes that are related to Alzheimers. In this way, a clinician user B is able to customize a therapy and treatment plan that will be most beneficial to the subject user A.

In use, the algorithms can be implemented into the above architecture by implementing the foraging/marginal value optimization game on a remote application device 20, such as a mobile or smart phone. To provide a measure of any mental health state, such as anxiety, the system would be configured to be utilized in the following manner:
1. The subject user A places the EEG head accessory 12 (e.g., headband with electrodes) on their head and begins a program on the remote application device 20 that activates the EEG head accessory 12. Alternatively, or additionally, the EEG head accessory 12 may include a power switch or control.
2. In the program, the subject user A can initiate a mental health or anxiety measurement by starting a gamified algorithm or decision-making game 31.
3. The remote application device 20 can time stamp the EEG data at the start of the game 31 to denote EEG data collected while the game 31 is being played by a subject user A. The time stamp can also include data from other sensors such as a heart rate sensor, temperature sensor, or respiration sensor.
4. While the subject user A is playing the foraging game 31 on the remote application device the EEG electrode head accessory 12 measures the EEG data of the subject user A and sends it to the remote application device 20.
5. The remote application device 20 can simultaneously or temporally send or transmit the raw EEG data to the data-platform 30.
6. The remote application device 20 can also send or transmit the results of the foraging game 31 or task, which can be considered non-EEG data. The raw EEG data and the results of the game 31 or non-EEG data can be transmitted separately or combined and transmitted together to the data-platform 30.
7. In one embodiment, the remote application device 20 is also able to compare the raw EEG data and the results of the game 31 or non-EEG data and produce a report (which can be in the form of a graphical display on the mobile device) that can be transmitted to a third-party user B such as a physician and/or displayed to the user B controlling the remote application device 20.
8. The data (raw EEG data and/or non-EEG data) received by the data-platform 30 can be post-processed. For instance, by filtering and steering the raw EEG data to measure the frequency power spectrum of the anterior cingulate cortex and other implicated brain regions. This data is then combined with the timing results of the foraging game to create a measure of anxiety for the user.
9. If this is the first time the measurement is performed or first time performed within a time-period, the data-platform 30 categorizes the data as a baseline measurement.
10. If the user EEG data matches the anxiety templates and the game results show that the user spent longer than average in each foraging patch, then a result is produced designating the user A as having high trait anxiety. A score can be created that is a combination of the correlation result to the anxiety templates and a magnitude deviation from the population average time in the foraging patch.
11. If the user EEG data matches the anxiety templates to a high degree and the game results shows that the user spent a shorter amount of time than the population average in each foraging patch, then a result is produced designating the user as having state or stress anxiety (e.g., anxiety response to a particular event). As described above, a score can be created that is a combination of the correlation to the anxiety templates and the magnitude deviation from the population average time in the foraging patch.
12. Subsequent measurements are compared to the baseline measurements to trend a user's A anxiety over time.
13. The results for the analysis and any trending information can be stored in the remote application device 20, and/or are sent from the data-platform 30, back to the remote application device 20 and the application or program 31 to be displayed to the subject user A.
14. The results can be made available to a third-party user B, such as a clinician, in real-time or may be accessed by a third-party user B when the subject user A and third-party user B meet. The third-party user B can access the results or output data points on any device that is able to connect to the data-platform 30, such as a mobile device or a computer able to access the provider portal 40 connected to or in communication with the data-platform 30.

### Monitoring in a Group—Synchrony

One of the many benefits of the present invention is its ability to monitor a group of individuals or subject users A. One example includes the monitoring of a particular state of the groups mind or body. Various states can be monitored, including but not limited to their mental state or health, behavioral state, and health state to name of few. The monitoring of the states is important to be able to monitor how a team works together in various situations and circumstances as will now be described. The system and methods of the present invention can simultaneously monitor, collect, and synchronize EEG data and/or non-EEG from a group of individuals. One advantage of the present invention is that the system 10 includes the ability to analyze the synchrony of a group of individuals. Synchrony is based upon the idea that group dynamics, teamwork, and human response can be better told and measured by viewing the EEG response of multiple people, users, or subjects at the same time. The system and methods 10 of the present invention can use the group's EEG data to determine and understand the group's response to stimuli, which could be an image such as a picture or a video; it could also be an event that the group is experiencing together, including but not limited to a concert, a movie, a gathering, or an altercation or engagement such as experienced by law enforcement or the military. The system and methods of the present invention can use individual user data and group data to monitor a state of the group or the group dynamics, such as a degree of teamwork, group and individual fatigue, and level of group aggression to name a few.

The system and methods 10 of the present invention can collect and use one or more synchrony datapoints in combination with group user EEG data and/or non-EEG data to monitor, analyze, and control or advise a group of users A. Synchrony datapoints comprise time, motion, and geography or location. The present invention can use the synchrony datapoints to synchronize the EEG data and non-EEG from participants to understand individual and group responses to a particular stimulus such as viewing a video, participating in a game, watching a speech or other event, engaging an altercation.

Geographic synchrony datapoint, when combined with the EEG data and/or non-EEG data allows the system of the present invention to monitor and analyze individual and group user states as they move amongst the stimulus, for example, moving around a manufacturing plant, or through a conference or expo, or when driving in traffic, or moving around a battlefield. Geographic synchrony datapoints when combined with the EEG data and non-EEG data will help to automatically show the response of multiple users A to a stimulus at a certain location. For instance, examples include:
Measuring fatigue response to a certain manufacturing process or activity;
Observing a distraction from drivers on public streets to show locations at higher risk for accidents; or
Observing the engagement of individuals to marketing displays or conference booths.

As illustrated in FIG. 1B, the system and methods 10 of the present invention include a one or more positioning systems 50 such as cell towers or a global positioning system “GPS” that can be incorporated into or in operative communication with any components of the system 10 such as the EEG head accessory 12, the remote application device 20, or a third-party GPS device, such as a smart watch or other geo-location devices. Any system 10 device or component can communicate with the positioning system or network 50.

### Time Synchrony

Continuing with FIG. 1B, the system and methods 10 of the present invention can synchronize multiple EEGs through the remote application device 20, a cell phone, or other wireless beacon broadcasting a standard time. In this example embodiment, time synchronized datapoints can be collected and synchronized between multiple users A by synchronizing with broadcast times and clocks on each user A. The time synchronized datapoints can be transmitted through the remote application device 20 or directly from the EEG head accessory 12.

Steps for establishing or collecting time synchronized datapoints using cellphone-based transfer comprises:
1. Remote application device 20 receives time from the internet, cellular, or gps network 50.
2. Optionally, the clock on the EEG head accessory 12 (e.g., headband) synchronizes to the clock of the remote application device 20.
3. EEG data is collected from EEG head accessory 12 and stored on the remote application device 20. The data can be time-stamped based upon the internet or broadcast time signal. A digital time can be interwoven in the EEG data stream periodically transmitted with the EEG data. If the latency of the EEG data transfer between the EEG head accessory 12 and the remote application device 20 is too large or variable, then the time synchrony datapoints and/or time stamping of data and/or datapoints can happen directly within the EEG head accessory 12.
4. Time-stamped data and time synchrony datapoints can be sent over the internet to the data server or data-platform 30.
5. On the data-platform 30, data from multiple users A is collected asynchronously or synchronously. With the time information, one or more users or third-party users can select an individual, group or subgroup of EEG data signals for synchronization. The remote application device 20 or data-platform 30 will align the EEG data signals and any non-EEG signals (e.g., responses of any data processing algorithms) and combine them in time based upon the received time synchrony datapoints.
6. In this manner, the data synchrony from multiple users A is obtained easily and automatically.

### Geographic Synchrony

The system and method described above can be augmented by instead of using time, GPS (global positioning system), Wi-Fi location data, or any type of location service or network 50 that is instead utilized and interwoven with the EEG data and non-EEG data. This places a location and/or timestamp on the data and allows an automatic synchronization by location.

### Geographic and Time Synchrony

The system and methods 10 of the present invention can also combine geography and time synchrony datapoints to obtain additional information. In this manner, individual and group EEG data responses at one or more times and/or at one or more locations can be established.

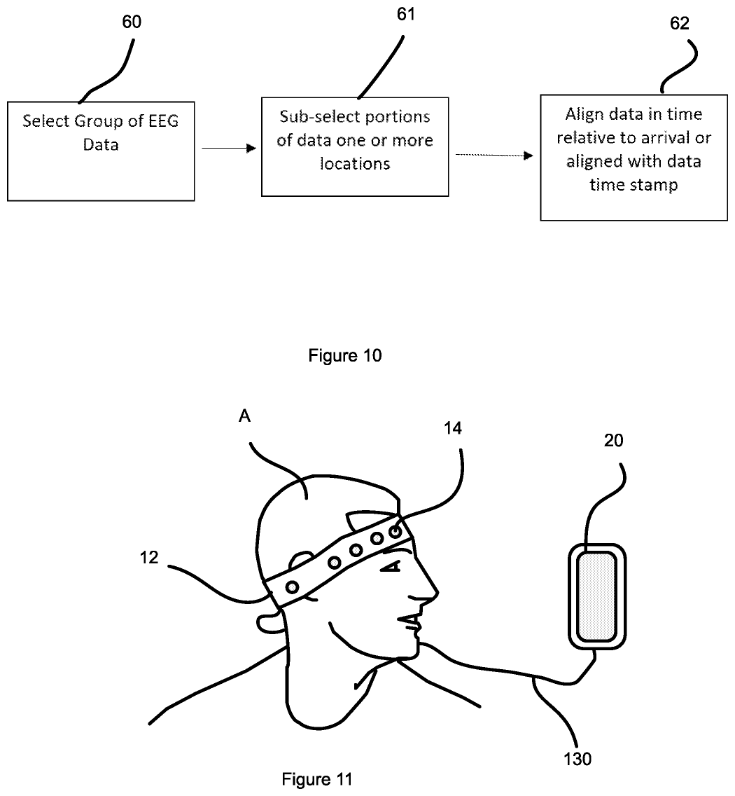

As illustrated in FIG. 10, the system and methods 10 can obtain select segments of EEG data and/or non-EEG data 60 (e.g., responses from applications interacted on the remote application device 20) from a single user at multiple time points where a stimulus would occur. A monitor user B is able to select a sub-set of data and one or more locations 61. The monitor user B with or without the system 10 is then able to align the data in time relative to the arrival or data time stamp 62.

In use, combining geographic and time synchrony datapoints with EEG data and non-EEG data can be implemented in the example situations:
Arrival of an individual to a certain location, such an employee arriving at the beginning of a manufacturing shift to conduct a certain process: Geographic and time synchronization datapoints can be combined with EEG data to allow automatic trending of fatigue or other cognitive or emotional responses over time.
Individuals or groups at a trade show observing a presentation that occurs at a preset time and location: The system 10 of the present invention allows for automatic selection of EEG data responses to a presentation.
Time on the job fatigue or distraction: this would allow trending fatigue or distraction over time at a particular or job on a ship or aircraft.
Training: EEG data and non-EEG data responses of a student (e.g., pilots in a training simulator) can be observed to observe responses to emergency situations. Time synchrony datapoints can be combined EEG data and non-EEG data to observe changes through the training.
Driving: distraction of drivers on a road at certain geographic locations. Timestamp would further allow determination of distraction based upon the time of day or night.

### Time Synchrony to Media

In another example embodiment of the present invention, as illustrated in FIG. 22A, EEG data and non-EEG data can be time or geography synchronized to a piece of media 70 shown on a device or in a theater. In this way, EEG data and/or non-EEG data responses of an audience or group A can be synchronized with time or geography synchrony datapoints to monitor a movie's 70 appeal or popularity and thus an indicator of how well it will do at the box office. The system and methods 10 can also be used with a group of users A to understand the appeal of a particular commercial, product, media, personality, etc. It can also be used to determine how successful a particular commercial or product will be in a particular geography, at a particular time of day, or during a particular tv show or sporting event.

The system and methods 10 of the present invention can utilize a media's 70 run time to establish a time-stamped datapoint. This feature allows the system 10 to be used remotely in a user's A home or in remote movie theaters. Users A can be sent or mailed EEG head accessories 12 that are able to read user's A EEG responses while they are viewing the media 70. All of the EEG data and non-EEG data can be collected from users A around the world and transmitted to the data-platform 30 for processing. The data-platform 30 can generate a report that can then be used by movie and commercial producers, product manufacturers, marketing companies, and the like, collectively B. The system and methods 10 of the present invention allow these third-party users B to obtain a physiological understanding of the potential success of a product (e.g., movie, commercial, or product) before investing in large scale manufacturing or distribution.

The present invention is able to use the data-platform 30 to combine or interweave the user A EEG data and non-EEG data with the media 70 viewed while collecting the EEG and non-EEG data. A third-party user B is then able to correlate the EEG data response and the non-EEG data with specific points in time of, or specific events occurring within, the media 70.

### Media Screening and Analytics

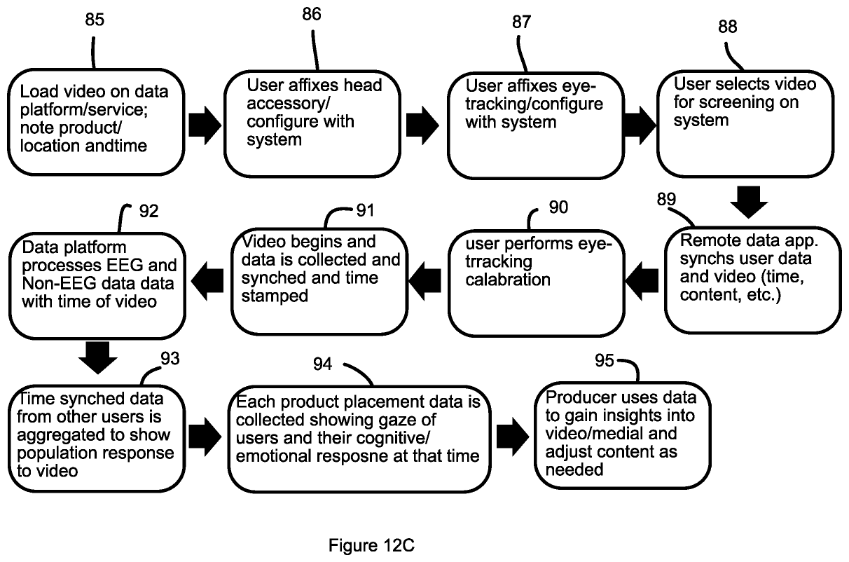

As illustrated in FIG. 12B, the EEG platform or system 10 of the present invention can be used in a variety of applications including healthcare, industrial safety, and business marketing. The EEG data collected by the system 10 can directly measure cognitive information of users A such as excitement, engagement, distraction, and positive and emotional states such as joy and sadness. Because of this, EEG data of the system A can provide a much more accurate and faster ways to measure the impact of a person experiencing media, such as video or audio, as well as printed material. The system A is able to provide valuable data related to preview testing, such as a film screening, to determine prior to release the impact of film or a series of film sequences.

Further, the system 10 can be utilized in the development of user interfaces in a variety of applications from computer software to aircraft pilot interfaces, to automobile driver dashboards. By measuring the EEG signals and the results data of the system 10 in time and in the presence of various stimuli or tasks, system designers can optimize screens and interfaces to minimize fatigue and distraction.

In all these applications, the EEG signals of the present invention can be merged with data from other sensors, such as heart rate or galvanic skin response to bolster the accuracy of the physiological cognitive response. Further, when combined with an eye tracking device 72 (see FIG. 12A), either in the form of eye-tracking glasses or a camera system embedded into a computer, mobile device, or nearby a television, eye position data can be used to determine which attribute of the media 70 provides a cognitive response. For instance, eye tracking data can be used to understand whether a user or viewer A is looking at a particular product placement in a film and the EEG then used to determine whether this creates a positive or negative emotion associated with the product. In the case of user interface testing, tracking of the eyes can determine how long the user A scans the interface prior to selection a particular function or if a particular alert or function creates a distraction.

As described above, the system's 10 architecture comprises an EEG head accessory or headband 12, a remote application device 20 having a mobile application 31, and a data-platform that comprises servers and other monitoring devices. For media screening, the system 10 includes a film screen, a television, or other monitor 70 to view video and/or audio playback. It is possible that that the monitor 70 showing the media is the same as the mobile device interfacing with the EEG head accessory 12 and the camera for eye-tracking is also integrated with that device 70.

As illustrated in FIG. 12A, the data transfer for a media screening application includes all sensors integrated together within the remote application device 20 prior to being sent over the internet to the data platform 30, however the concepts would be employed similarly if all sensors sent information individually to the data platform 30.

The system 10 of the present invention is architected such that it can be used in a remote traditional theater screening location or could be performed remotely in a user's home without significant setup or equipment burden. As a video 70 is started and played by the user A, physiological data is collected and sent to the data platform 30 for aggregation and analysis. Further, data between multiple users A in multiple locations can be aggregated together to provide a high data number analysis without holding a single, large in-person screening.

FIG. 12B shows a flowchart of a remote video screening process. A first step 74 comprises a producer loads a media onto a data platform 74 that can be downloaded by a user A. The next step 75 entails a user A affixing the head accessory 12 to their head and connecting it to the remote application device 20. If an eye-tracking appliance 72 is used the user can affix it to their head in step 76. In the next step 77, a user A is able to select the media to be viewed. In step 78 the remote application device 20 synchronizes the media and the streaming data. Optionally, in step 79, the remote application device 20 is able to synchronize eye-tracking data with the other data. In step 80, all of the collected data is timestamped in relation to the media being viewed and sent to the algorithm platform 30 for analysis. In step 81 the algorithm platform 30 performs the analysis of the data and in step 82 it is able to synchronize it with data from other individuals being monitored. In step 83 trends and relevant data points are collected into a report that is then given to a monitor user B in step 84.

Some examples of insights that can be developed from the video and media screening by viewing deviations in specific EEG signals:
Overall cognitive/emotional response at the end and throughout the media viewing
Time points creating significant stress
Engagement of the viewer during the media. This can be trended and shown to the content producer as a time plot either in synchrony with media or as an analytics plot. In this manner, the content producer would know if the users lose engagement at any point during the media/film/video.
Degree of synchrony between all users. This average response is indicative of the strength of the cognitive response at various time points.
Degree of EEG synchrony between users watching the same video at the same time and location. Synchrony between users has been shown to be a positive predictor of future box-office sales.
Difference in cognitive and emotional state from beginning to end of video—provides a normalizing of cognitive data between user/viewers
Separation and analytics of user/viewer response by demographics such as gender, age, location, etc. This can be selected or highlighted automatically by the artificial intelligence algorithms employed on the data platform.
Separation and analytics of user/viewer responses to various media changes such as alternative endings, alternative audio tracks, deleted scenes, etc.

Note that the above process and algorithms can be performed on pre-recorded videos such as movies, but the process and algorithms would perform equally well on live-streamed events such as political debates and athletic events.

### Product Placement Analysis & Analytics

In the case of the film industry and TV shows, a source of income for the productions are often revenue from product placements. Examples are the use of branded drinks and food, choice of automobiles and transportation brands, fashion ware, retail establishments, etc. To better monetize these placements, the system 10 can be utilized to show the value of various placements.

FIG. 12C shows a flowchart of a remote product placement to measure the value of a product using the system 10 of the present invention. A first step 85 comprises a producer loading a media containing a product onto a data platform that can be downloaded by a user A. The next step 86 entails a user A affixing the head accessory 12 to their head and connecting it to the remote application device 20. If an eye-tracking appliance 72 is used the user can affix it to their head in step 87. In the next step 88, a user A is able to select the media with the product to be viewed. In step 89 the remote application device 20 synchronizes the media and the streaming data. Optionally, in step 90, the remote application device 20 is able to synchronize eye-tracking data with the other data. In step 91, all of the collected data is timestamped in relation to the media with the product being viewed and sent to the algorithm platform 30 for analysis. In step 92 the algorithm platform 30 performs the analysis of the data and in step 93 it is able to synchronize it with data from other individuals being monitored. In step 94 eye-tracking and other data pertaining to the product are collected into a report that is then given to a monitor user B in step 95.

Some examples of insights that can be developed about the product placement by analyzing the EEG and eye tracking data include:
Percentage of viewers or users whose eyes looked directly at a particular product placement
Amount of time viewers or users' eyes remained affixed on product
Cognitive and emotional response of viewers or users at the time viewers or users' eyes were affixed on product, e.g., joy, sadness, distraction, etc.
Degree of synchrony between all viewers or users when viewing product placement. This average response is indicative of the strength of the cognitive response for each placement.
Degree of EEG synchrony between viewers or users watching the same video at the same time and location.
Subset analysis of user/viewer response by demographics such as gender, age, location, etc. This can be selected or highlighted automatically by the artificial intelligence algorithms employed on the data platform.

All of the above analysis combined to produce a product placement “score” that would indicate the amount of time viewers noticed the product and the degree they felt positive about it. These scores can then be used to monetize the value of a product placement within a film or media content.

### Operations Monitoring

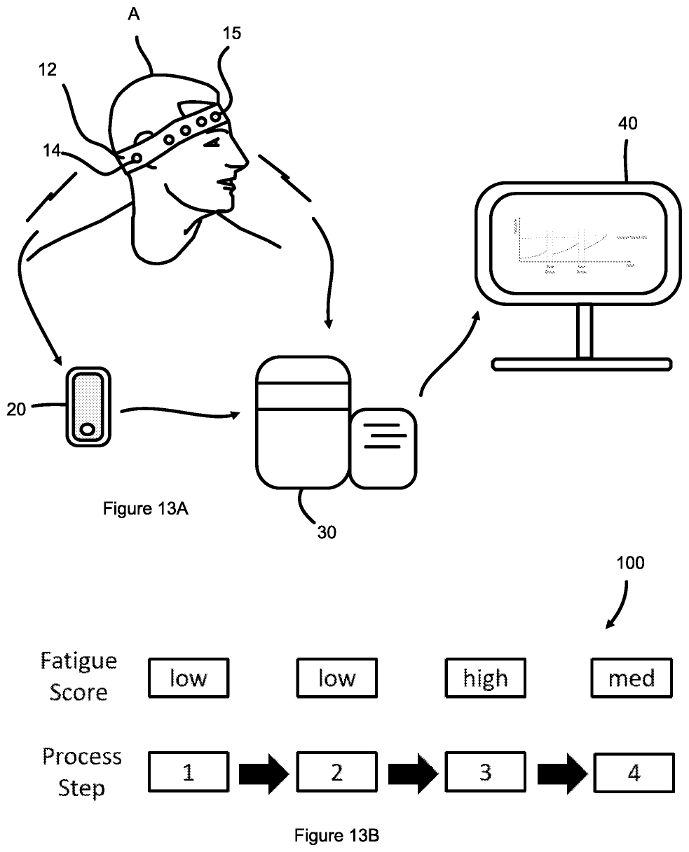

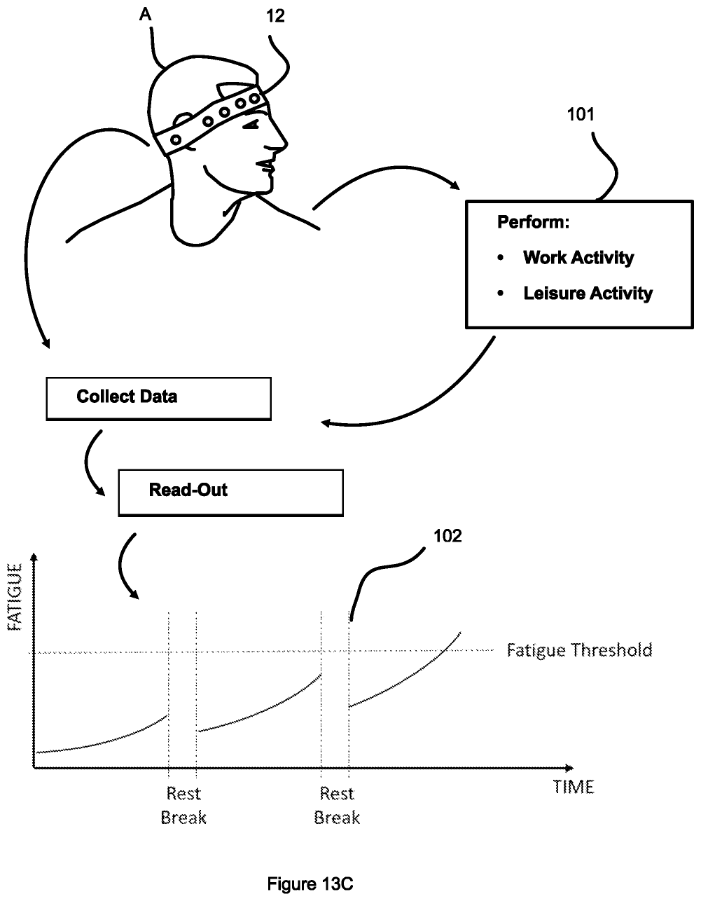

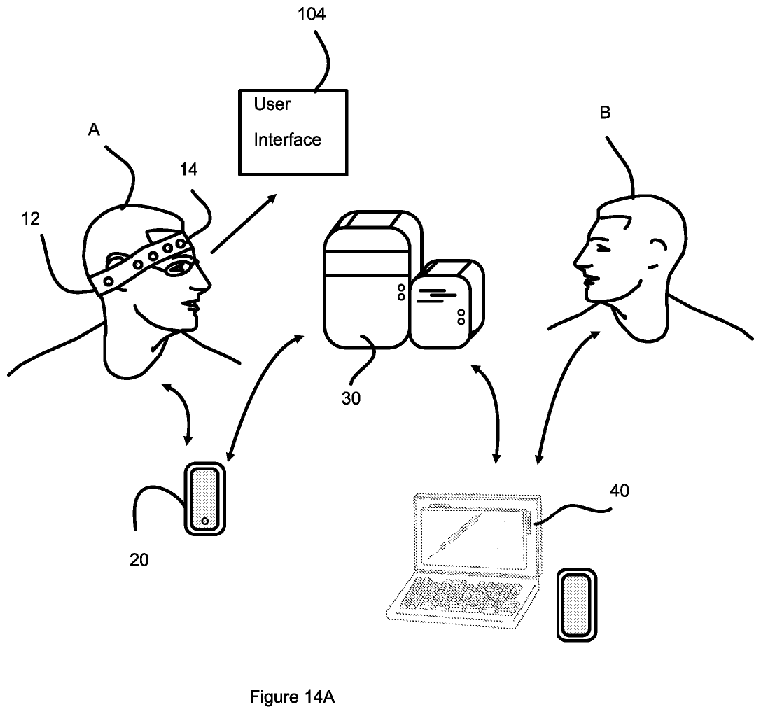

In FIGS. 13A and 13B, the system 10 is utilized to monitor a subject user A performing a task. In this section, a subject user A is referred to as an operator A and can refer to an operator in any process, for example a manufacturing operation in a manufacturing or industrial plant, a sailor on a civilian or military ship, or any other activity that requires a human to complete a task.

Studies have shown that the majority of workplace accidents are caused by fatigue, distraction, and poor training. The systems and methods 10 of the present invention, utilizing wireless and high-quality EEG electrodes 14, is able to monitor and trend these factors. The present invention is also able to generate a graphical output that identifies, areas of risk. Examples of graphical outputs of the present invention include creating a heat-map of the highest area of risk, devices or machines of highest risk, and times of highest risks. The system 10 is able to determine the following, in addition to others:
Average time to operator fatigue performing a task;
Distractions associated with plant's environment;
Effectiveness of training and the amount of time it takes a task to become well-learned or rote after training; and
Impairment of an operator from fatigue or chemical substances.

### Process Steps for Manufacturing Optimization

Referring to FIG. 13C, all operators A participating in manufacturing optimization wear an EEG headband or head accessory 12. An operator A then performs a manufacturing process step 101. Fatigue, concentration, distraction are measured over time and trended 102 as an operator A performs the manufacturing step. In some instances, operators A may need to have a baseline measure of fatigue to calibrate the EEG system 10 for their personal situation. Fatigue trends 102 are compared to a threshold to determine when the operator A hits a point of mental fatigue. This data is then used to optimize the process steps for the human operator A. Examples redesigns include but are not limited to the following:
Redesign the process to limit or reduce mental fatigue
Plan rest breaks at optimal time
Match operators with the best processes for their abilities
Real time determination of whether an operator is overly fatigued to perform a process
“Lock-out” a safety critical process if the operator is fatigued or distracted
Trend operator frontal lobe activity in the days/weeks after training to understand the training effectiveness
Trend fatigue over weeks to determine whether fatigue improves with process knowledge.

The data gathered be presented geographically within a plant to show “hot spots” or processes that generate the highest fatigue. This could trigger the rotating of operators A. In this manner, each process would be given a fatigue score 100, as illustrated in FIG. 13B, both in trending and in real-time from operators performing the process.

As discussed in more detail above, time stamp and location data be automatically generated based upon signal triangulation from Bluetooth, Wi-Fi, or other signal from the EEG headband 12 or the user's remote application device 20 or cell phone.

Manufacturing Optimization with Contact Tracing

The previous describes the system of the present invention being used for optimizing a manufacturing plant, a ship or any steps in a process based upon EEG data. This system 10 also comprises a suite of sensors and monitors to enable manufacturing operations to function safely during a pandemic scenario.

Because the EEG headband or headgear 12 is in contact with the forehead, non-EEG sensors 15 such as a temperature sensor, can be incorporated therein to monitor worker or operator's A vitals such as temperature for early warning of the onset of illness. This is of particular importance to identify employees A that may pose an infection risk to the rest of the employees A. By being able to detect an elevated temperature a company can quickly isolate the employee. The temperature sensor A can also be used to detect increases and decreases in body temperature for any user A that spends time outdoors. In hot environments the system 10 can detect hyperthermia quickly and in cold environments the system is able to quickly detect hypothermia. Allowing the user or an employee A to seek an appropriate environment.

The headband 12 of the system 10 can also include an accelerometer sensor 15 to measure movement and activity. The accelerometer sensor 15 can measure a user's A movement or lack of movement. This is important in healthcare settings where movement or non-movement of a patient must be monitored. The accelerometer sensor 15 can also be used to monitor a user's A wellness by detecting and measuring their physical activity. For employees A on the job it can be used to determine if they are obtaining enough or any physical exercise during their shift and day.

Signals from the headband 12, a user's remote application device 20 such as their cell phone, or any of the other sensors 15, can additionally be used to show proximity of the users A or operators A to one another. This is useful to help maintain social distancing and can be expanded to show contact tracing in the event of a pandemic virus breakout. This system 10 is able to help update those potentially infected, provide data to allow healthy workers to keep a plant, store, or office operational, and provide a faster time to notification of illness.

### User Interface Testing

Turning to FIG. 14, the system 10 can also be used for user interface testing. In this application the media film content 70 can be replaced by a user interface 104 for software, machine operation, etc., to gather analytics in order to measure and optimize the user A and operator experience. This can be performed in a real-time evaluation or within a simulated environment, such as an aircraft simulator. FIG. 14 shows a diagram of the system architecture. Similar to the to the media screening applications above, the system collects a recording of interactions with the user interface along with the physiological data.

The system 10 of the present invention is able to collect one or more recordings of interactions with one or more user interfaces along with the physiological data of the users A.

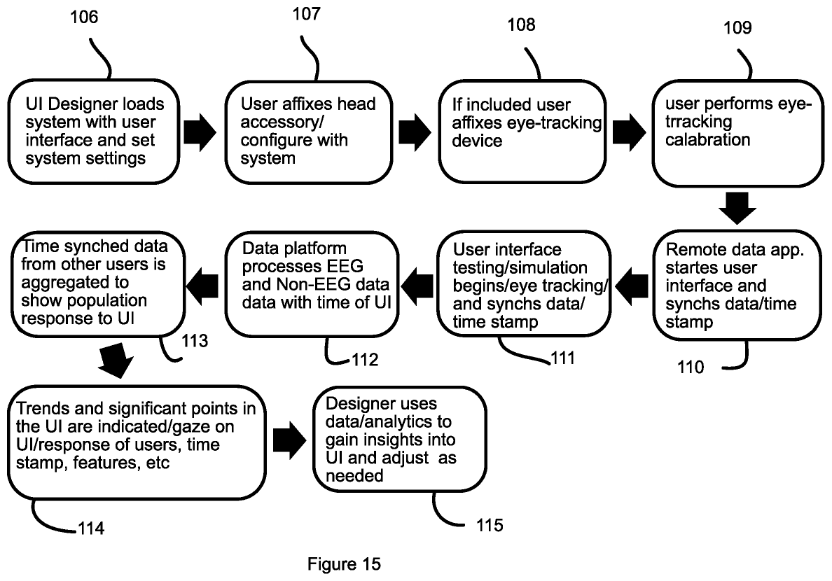

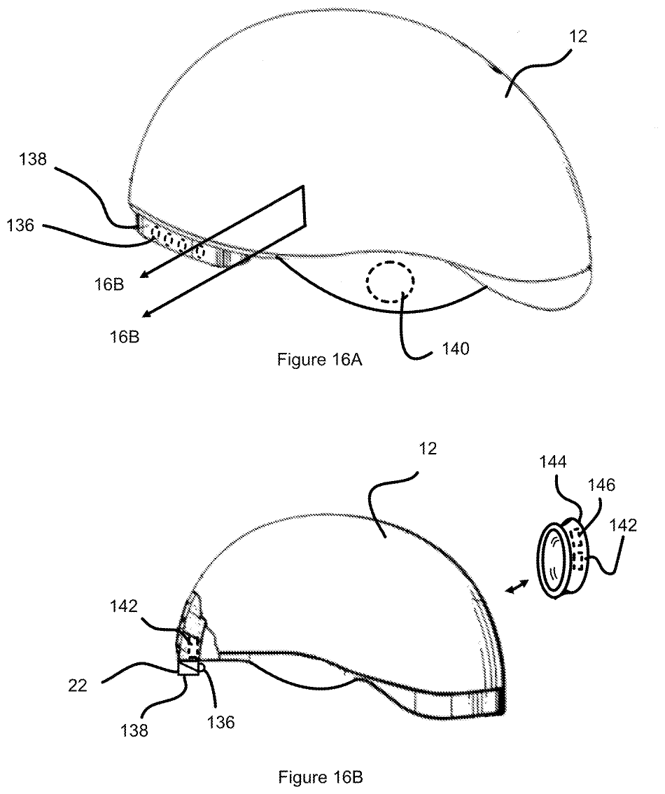

FIG. 15 shows a flowchart of a user interface testing to measure the value of a user interface using the system 10 of the present invention. A first step 106, comprises a UI designer loading a user interface onto a data platform that can be downloaded by a user A. The next step 107 entails a user A affixing the head accessory 12 to their head and connecting it to the remote application device 20. If an eye-tracking appliance 72 is used the user A can affix it to their head in step 108. In the next step 109, a user A eye-tracking is calibrated. In step 110 the remote application device 20 starts the user interface or simulator and the data is timestamped and/or synchronized. In step 111, the user begins to use the user interface or simulator and all of the data is synchronized and/or timestamped. In step 112, all of the collected data is timestamped in relation to the user interface or simulator and sent to the algorithm platform 30 for analysis. In step 113 the algorithm platform 30 performs the analysis of the data and is able to synchronize it with data from other individuals being monitored. In step 114 eye-tracking and other data pertaining to the user interface are collected into a report that is then given to a monitor user B in step 115.

Some examples of insights that can be developed about the User Interface by analyzing the EEG and eye tracking data:
Distribution of and percentage of gaze time on various areas of the user interface.
Amount of time users' eyes scanned prior to implementing a particular function.
Amount of time between triggering an event, such as an emergency alarm in a simulator, and the reaction by the user.
Degree of synchrony and average variance in time between all users when implementing a particular function or responding to an event.
A distribution and measure of the average amount of time users spent in particular user interface functions.
Cognitive response of the user to particular events and functional implementation. For example, measure of the degree of engagement, distraction, and stress during the use of the product or interface
Synchrony of cognitive responses between users in response to particular events
Fatigue profile of users over time and functional activities. This could measure both the specific fatigue introduced by a particular event or function as well as provide an overall measure of the cognitive load of the particular events or user interface manipulation.
Provide a distribution of time between users until cognitive impairment becomes likely from fatigue.
Subset analysis of user/viewer response by demographics such as gender, age, location, etc. This can be selected or highlighted automatically by the artificial intelligence algorithms employed on the data platform.
Automatic highlighting of particular functions that trigger an excessive distraction or stress response.
Automatic highlighting and segmentation of particular events that may cause a critical system failure, for example, a crash of an aircraft in a simulator.

### Military Application

As briefly mentioned above, the system and methods 10 of the present invention is ideally suited for police and military use. However, for military use digital security is of the upmost importance and wireless data transfer can be a data security risk. Not only could biological data be intercepted, but the wireless transfer could create and avenue for bad actors to enter an otherwise secure network. Because of this many military facilities dealing with confidential information, such as a Sensitive Compartmented Informational Facility (SCIF), cannot allow wireless systems within their facility.

This creates an issue for use of a cognitive wearable such as an EEG in a secure environment. Use cases such as monitoring operators for fatigue, measuring cognitive response to situational events, and improving human performance in sensitive and stressful environments could become more difficult. Not only would the real-time data transfer not be possible, a user A would not be able to interact with a wearable function through a wireless data link, such as Bluetooth, to a mobile device. In these situations, an alternative architecture is needed for data collection and monitoring of device functionality. The present invention's architectures and designs overcome these challenges while maintaining ease-of-use of the system.

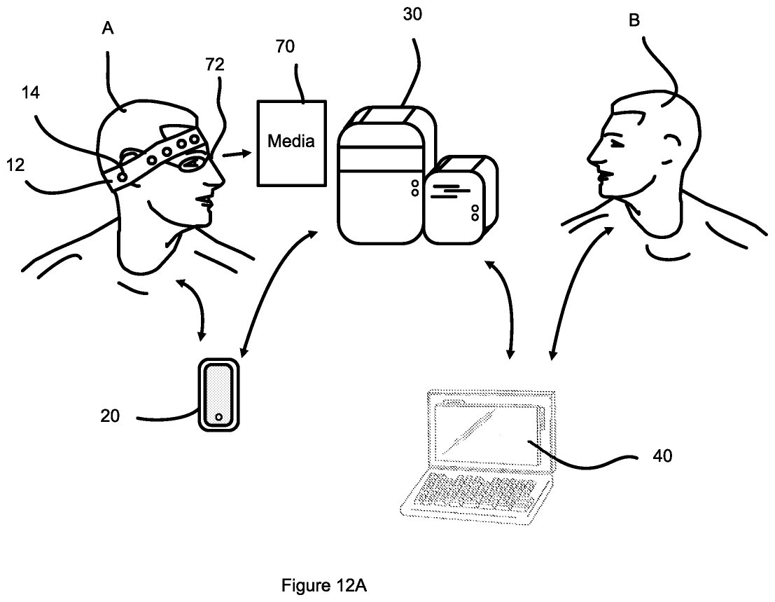

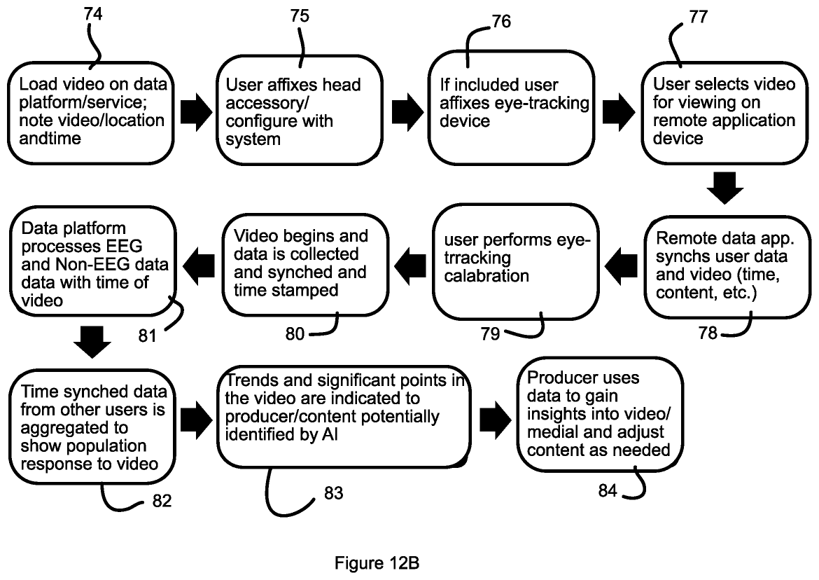

As illustrated in FIG. 11, in this embodiment of the present invention, instead of a wireless link to a processing or mobile device 20, there is wired connection 130 to the processing or mobile device 20 or a storage platform or data collection device carried on the user. The data storage device can be connected to the EEG head accessory 12 by wires, wirelessly, near-field wireless communication. In the wireless configuration, the data transmission distance is very short (e.g., a several inches to one or two feet). Data encryption can also be used to protect the data.

Instead of a tether 130, the EEG data is stored locally in the headband 12 electronics. The data could be stored in memory or on a removable data storage device such as a flash SD card. In this configuration, the EEG head accessory or headband 12 includes an on-board real-time clock. The clock data is added to the EEG data stream stored locally on the storage device in order to provide a timestamp to certain events and evoked responses.

### Leads-Off Indication

One challenge with an untethered, local-storage EEG device such as the head accessory 12 is starting the device 12 in operation, monitoring the status of the power supply or battery, and monitoring the connection between the sensor electrodes 14 and the user's A skin. The system 10 employs a leads-off indicator that notifies the user A when a particular electrode 14 is not making strong electrical contact with the user's A body. Because the mobile, wireless EEG head accessory 12 is on the user's A head, the user A cannot easily see an indication of lead status nor battery status. These types of electrode 14 testing processes are performed on wireless and/or tethered systems 10, thereby resolving potentially negative effects (e.g., security or data integrity breaches) that may occur if not performed.

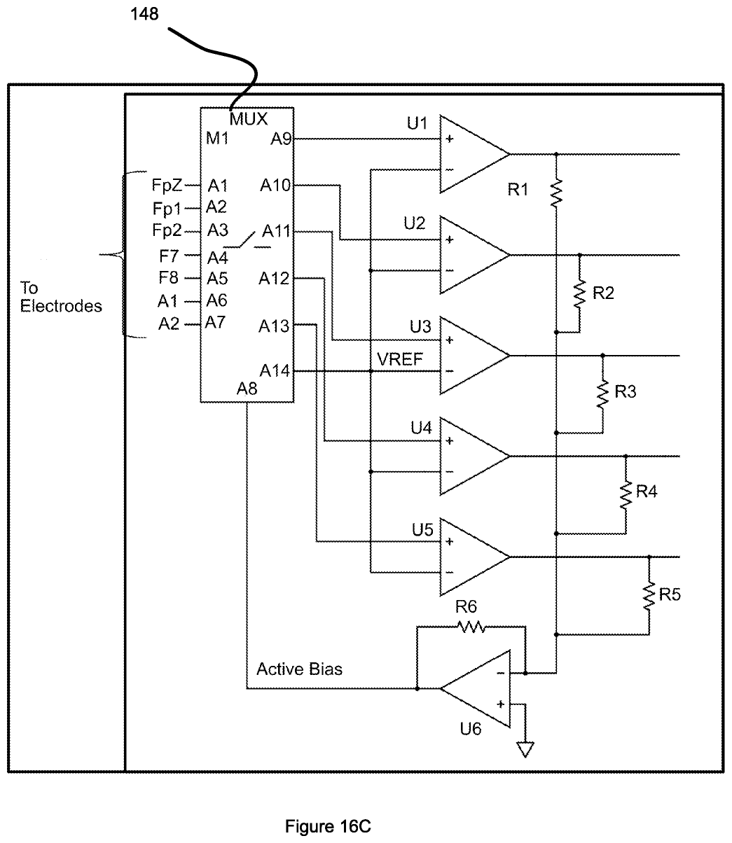

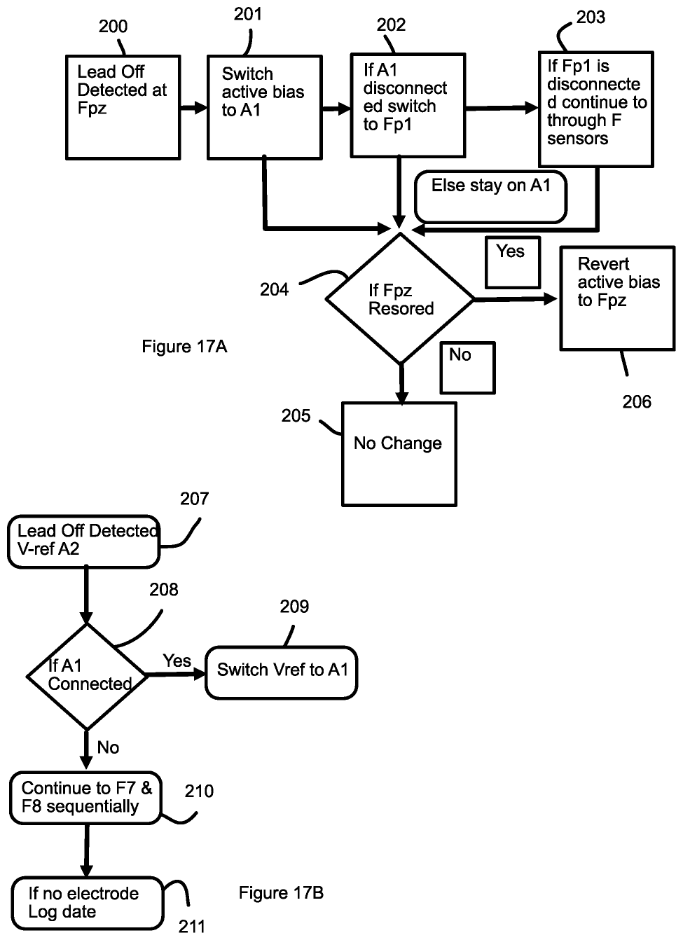

To overcome these limitations, a series of visual indicators or lights 136 are employed on the EEG electrode appliance or headband 12 to provide an indication of the status of the electrode 14. As illustrated in FIGS. 16A and 16B, these indicators 136 can be positioned in a variety of configurations. For example, they can be positioned opposite each electrode 14 or lined up together such that the user A can view the lead electrode 14 status by monitoring the indicators 136 with a reflective surface such as a mirror. The visual indicators 136 can also be operatively located on a user's helmet 12 above their eyes on a small visor 138 that extends down from the helmet 12. In this configuration the user A can determine a status by a light location with respect to the helmet 12 or by a status lights color. As illustrated in FIG. 16B, the visual indicators 136 can be positioned on an inner surface of the helmet 12 and/or visor 138 such that it is only visible to the user A.

Alternatively, an audible subsystem can be included that can emit an audible warning to the user A of a disconnected electrode 14 or lower power. The audible warning can be an audible beep or different sound. The system 10 includes different sounds to indicate different status, for example, a lower batter or a malfunctioned electrode 14. As illustrated in FIG. 16A, the audible system 140 comprises an audio emitting device such as a speaker that can be incorporated into the head band or helmet 12. The audio system 140 can be a voice instruction recording to notify the user A of the state of a component of the system 10 (e.g., a disconnected electrode). This audible system 140 could be either manually or automatically triggered. The status of a particular component of the system 10 can be immediately relayed to a third-party user B such as an equipment technician that can use the user monitor or system 40 to locate the user A and either advise the user A how to correct the indicated issue or make their way to the user A to correct the malfunction. The third-party user B can also include a military strategist that is able to call the user A back from engagement while simultaneously moving one or more other users A to replace the retreating user A.

In another example embodiment of the present invention, the system 10 may include one or more vibratory devices 142 or transducers placed within or on the EEG electrode appliance or headband 12. The vibratory devices 142 can be placed opposite to or proximate to each electrode 14 thereby enabling a user to feel the vibration at a particular electrode 14 that is experiencing an issue. The vibratory devices 142 can also be placed in any location that a user A is able to sense their emitted vibrations.

In an example embodiment, as illustrated in FIG. 16B, a remote device 144, such as a wristband, can be used with one or more vibratory devices 142 incorporated into it. Each electrode 14 can correspond to a particular vibratory device 142 on the wristband. In another example embodiment, the vibratory device 142 is configured to vibrate in different patterns with each pattern correlating to a particular electrode 14 or other system 10 component. In this way, the user A can determine which electrode 14 is malfunctioning.

The wristbands 144 can include a wireless transmitter and/or receiver 146 that communicates with the other components of the system 10 of the present invention. The wristband 144 can be selectively limited or configured to receive leads-off information only to notify the user A. It can also be selectively configured to send the leads-off information to a third-party user B that is monitoring one or more users A wearing the system 10. The ability to select different receipt and transmit states allows the system 10 to control the leads-off information.

Not being able to view the status of the electrical EEG signals and leads-off-indicators can create the situation where poor data is recorded without the user A or B being knowledgeable that an adjustment is needed. In these cases, poor data quality at one electrode 14 could contaminate others during calculations or in the case of the active bias electrode 14 not making contact, contaminate all electrodes 14, providing poor and in the worst-case, unusable data.

Alternatively, to make the system 10 more robust, the system 10 can sense and denote in the data when electrode 14 impedance exceeds a threshold. In this case, the data would be demarked as leads-off, and the data from that electrode 14 would be discounted in the calculations. In the case of the active-bias electrode 14, in the system's 10 case located nominally at the FpZ position (see FIG. 3), a loss of this electrode 14 could contaminate all of the electrodes 14 and/or make it difficult to extract any signal.

Referring to FIGS. 17A, 17B, and 3, any changes from the nominal electrode 14 configuration would be included within the time stamped data such that these changes can be taken into consideration during subsequent EEG signal processing. To address a failure in the active-bias electrode 14, the system 10 of the present invention includes a backup electrode 14 switching subsystem that can automatically detect a malfunctioning electrode 14 and switch to either a backup active-bias electrode 14 or to designate another electrode 14 as the active-bias electrode 14. As illustrated in FIG. 16C, the system 10 includes a multiplexer 148 in the electronics making it possible to switch the active-bias electrode 14 to another position, thereby maintaining the integrity of the remaining electrodes 14 and the collected data. The system 10 is able to run the following example process:
In step 200, an electrode 14 is detected as being off
In step 201, the system 10 switches to active bias electrode A1.
In step 202, if the active bias electrode A1 is detected as being disconnected it will switch to electrode Fp1.
In step 203, if electrode Fp1 is detected as being disconnected the system 10 will cycle through the rest of the electrodes.
In step 204, if electrode Fpz is restored the system 10 will revert active bias to electrode Fpz
In step 205, if electrode Fpz is not restored it will make no change.

As illustrated in FIG. 17B, the system 10 is also able to run create and adjust feedback loops to account for any number of electrodes 14 and/or sensors 15 and their configurations (i.e., location of reference and common-mode feedback bias). The system is able to run the following process:
In step 207, an electrode A2 is detected as being on
In step 208, the system 10 determines if electrode A1 is on
In step 209, the system 10 switches Vreff to electrode A1 if step 208 is yes
In step 210, the system 10 continues to electrodes F7 & F8 if step 208 is no
In step 211, the system 10 creates a log date if no electrode is detected

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products. Moreover, features or aspects of various example embodiments may be mixed and matched (even if such combination is not explicitly described herein) without departing from the scope of the invention.

What is claimed is:

1. A field-wearable neural activity monitoring system wearable to monitor a neural activity of a remote user, the system comprising:
a head accessory assembly comprising:
a headband extending at least partially about a head of a remote user, the head band having an inner surface and an outer surface, the inner surface being positionable against a head of the remote user;
a sensor having an electrode surface and non-electrode surface, the sensor being removably coupled to a portion of the headband with the non-electrode surface being positioned against the inner surface of the headband; and
a mobile data collector in operative communication with the head accessory assembly to collect neural activity data of the remote user detected by the sensor.

2. The field-wearable neural activity monitoring system, of claim 1 further comprising a plurality of sensors coupled to a connector extending between and operatively coupled to the electrode surface of the plurality of sensors positioned proximate to the outer surface of headband.

3. The field-wearable neural activity monitoring system of claim 1 further comprising a neural activation event configured to initiate neural activity of the remote user.

4. The field-wearable neural activity monitoring system of claim 1 further comprising a remote computer in operative communication with the mobile data collector to receive and process a neural state of the remote user.

5. The field-wearable neural activity monitoring system of claim 3, wherein the neural activation event comprises a program stored on a remote device and operated by the remote user.

6. The field-wearable neural activity monitoring system of claim 3, wherein the neural activation event comprises an activity performed or observed by the remote user in real-time.

7. The field-wearable neural activity monitoring system of claim 4, wherein the remote computer is configured to synch neural activity of a plurality of remote users to establish a collective neural state of the plurality of remote users.

8. The field-wearable neural activity monitoring system of claim 1 further comprising a coupler member disposed between the headband and the sensor, wherein the sensor is easily replaced by the remote user.

9. The field-wearable neural activity monitoring system of claim 8, wherein the coupler comprises spaced apart first and second magnet members coupled to the non-electrode surface of the sensor, wherein the first magnet member is positionable proximate the inner surface of the headband and the second magnet member positionable proximate to the outer surface of the headband, whereby the senor is removably secured to the headband.

10. The field-wearable neural activity monitoring system of claim 1 further comprising random access memory coupled to the head accessory to receive and at least temporarily store the detected neural activity data, wherein mobile data collector is in wireless communication with the head accessory to collect the detected neural activity data from the random-access memory.

11. The field-wearable neural activity monitoring system of claim 4, wherein the mobile data collector encrypts the neural activity data until its transmitted to the remote computer.

12. The field-wearable neural activity monitoring system of claim 4, wherein the neural state detected comprises a normal state, an Alzheimer's state, a depressive state, an anxiety state, a bipolar state, an exhaustive state, a confusion state, or an altered mental state.

13. A field-wearable neural activity monitoring system wearable by a plurality of remote users configured to detect a mental state of the plurality of remote users, the system of each remote user comprising:
a head accessory extendable about a head of a remote user, the head accessory comprising;
a plurality of sensors configured to detect neural activity data of the remote user experiencing an event, each of the plurality of sensors being folded about a portion of the head accessory;
random access memory coupled to the plurality of sensors to at least temporarily store the neural activity data;
a program stored in the random access memory to control the plurality of sensors;
a power supply coupled to the head accessory and in operative communication with the plurality of sensors and random access memory;
a data collector in operative communication with the head accessory to collect the neural activity data of the remote users over time; and
a remote computer in operative communication with the mobile data collector to receive and process the neural activity data to determine a mental state of the remote users.

14. The field-wearable neural activity monitoring system of claim 13 further comprising remote device containing a program used by a remote user to elicit neural activity.

15. The field-wearable neural activity monitoring system of claim 13, wherein the head accessory has an inner surface and an outer surface and each of the plurality of sensors comprises an electrode surface and a non-electrode surface, the sensor being foldable about with the non-electrode surface being positioned against the inner and outer surfaces of the head accessory.

16. The field-wearable neural activity monitoring system of claim 13, wherein the event being experienced by the remote user is a virtual event.

17. The field-wearable neural activity monitoring system of claim 13, wherein the event being experienced by the remote user is a non-virtual event.

18. The field-wearable neural activity monitoring system of claim 13, wherein the plurality of sensors are removably coupled to a portion of the head accessory by one or more magnets.

19. The field-wearable neural activity monitoring system of claim 13, wherein the remote computer detects a neural activity state comprising a normal state, an Alzheimer's state, a depressive state, an anxiety state, a bipolar state, an exhaustive state, a confusion state, or an altered mental state.

20. A method of monitoring a mental state of one or more remote users, the method comprising:
providing a head accessory having one or more neural activity sensors configured to detect neural activity of the remote user;
providing a neural activity event to elicit neural activity from the remote user;
providing a neural activity data collector in operative communication with the one or more neural activity sensors to collect neural activity data of the remote user over time;
providing a remote computer configured to receive the neural activity data from the neural activity data collector and to process the neural activity data of the remote user; and
determining a mental state of the remote user by processing the experiencing the neural activity data in the remote computer.
